(19) United States
(12) Patent Application Publication
(10) Pub. No.: US 2019/0298295 A1
(43) Pub. Date: Oct. 3, 2019

(54) SENSING AND USING ACOUSTIC SAMPLES OF GASTRIC SOUND

(71) Applicant: Carnegie Mellon University, Pittsburgh, PA (US)

(72) Inventors: Ali Momeni, Pittsburgh, PA (US); George Loewenstein, Pittsburgh, PA (US); Max G`Sell, Pittsburgh, PA (US)

(21) Appl. No.: 16/316,977

(22) PCT Filed: Aug. 3, 2017
(86) PCT No.: PCT/US17/45249

§ 371 Date: Jan. 10, 2019

Related U.S. Application Data
(60) Provisional application No. 62/494,342, filed on Aug. 4, 2016.

(51) Int. Cl. A61B 7/00; A61B 5/00; A61B 5/16

(52) U.S. Cl. CPC A61B 7/008; A61B 5/0022; A61B 5/165; A61B 5/7246; A61B 5/7267; A61B 5/7203; A61B 5/6823

(57) ABSTRACT

Among other things, a sensor is configured to sense sound from a gut of a person and to output a corresponding acoustic signal. A signal processor has an input for the acoustic signal and is configured to output corresponding digital acoustic samples. There is a storage device for the digital acoustic samples. A processor is configured to apply the digital acoustic samples to a model to determine an aspect of a state of the person. A device is configured to present information about the determined aspect of the person's state to a user.

This application is entitled to the benefit of the filing date of U.S. provisional application 62/494,342, filed Aug. 4, 2016, the entire contents of which are incorporated here by reference.

### BACKGROUND

This description relates to sensing and using acoustic samples of gastric sound.

Substantial empirical research supports the commonplace intuition, often gained from direct experience, that there is a close connection between the mind and the gut. Psychological states and emotion-evoking situations produce noticeable reactions in gastric activity, and many chronic diseases afflicting the gut, such as ulcers and Crohn's Disease, can be triggered or accentuated by stress. Gastric disease is extraordinarily prevalent and costly. In the U.S. alone in 2004, the U.S. Department of Health and Human Services estimates, digestive diseases afflicted 60-70 million people and led to treatment costs totaling about $100 billion; peptic ulcers alone afflicted about 15.5 million and led to 358,000 hospitalizations and 3,000 deaths. Gastric conditions are a major source of misery, as well as cost, to society, and an important medical specialization, gastroenterology, focuses on their study, diagnosis, and treatment.

The human gut is regulated by a network of approximately 100 million neurons, clustered around the intestines. This “enteric nervous system” (ENT), which is sometimes referred to as the “second brain,” has both direct connections to the ‘first’ brain via the vagus nerve, and indirect connections via nerves, hormones and its role in the production of neurotransmitters. Signals in the vagus nerve flow in both directions, but those from the gut to the brain predominate over those traveling in the opposite direction, suggesting that the gut is an important source of signals to the brain.

While the human brain has received a tremendous amount of attention from researchers, the ENT has received far less attention. Although there is a field of research—neurogasteroenterology—focusing on the ENT, it has attracted very few researchers who, collectively, have so far have not produced a large body of research findings of broad significance. Psychogastroenterology is an even less developed field. Some gastroenterology clinics do provide patients with psychological counseling, instructions on how to reduce stress, or (most commonly) anti-anxiety medications, but scientific understand of the brain-gut connection is currently at a very early and primitive stage of development.

Progress in both fields has been slow, in large part due to a paucity of practical methods of measurement, and specifically the inapplicability to the study of the gut of the main methods that have revolutionized neuroscience. The main methods of neuroscience research: fMRI and PET scans, and EEG measurements, are all poorly suited for studying the GI tract for reasons of unreliability, intrusiveness, cost, and/or exposure of patients and/or research subjects to radiation.

### SUMMARY

In general, in an aspect, a sensor is configured to sense sound from a gut of a person and to output a corresponding acoustic signal. A signal processor has an input for the acoustic signal and is configured to output corresponding digital acoustic samples. There is a storage device for the digital acoustic samples. A processor is configured to apply the digital acoustic samples to a model to determine an aspect of a state of the person. A device is configured to present information about the determined aspect of the person's state to a user.

In implementations of this and other aspects, one or a combination of two or more of the following features may be present. The sensor includes one or more microphones or stethoscopes. There is a structure to be worn by the person and to support the sensor in an orientation to receive the sound from the gut. The structure to be worn by the person also supports a wireless transmitter. The model includes a predictive model. The model includes a Bayesian model. The aspect of the state of the person includes a gut state or an emotional state. The information presented to the user includes a visualization associated with features of the acoustic samples. The information presented to the user includes audiovisual representations of the determined aspect of the person state. The audiovisual representations include biofeedback for the user. The information presented to the user is representative of a diagnosis of the aspect of the state of the person. The information presented to the user is representative of a therapy for the aspect of the state of the person. The information presented to the user is representative of a prognosis for the aspect of the state of the person.

In general, in an aspect, sound is sensed from a gut of a person. Digital acoustic samples are stored representative of the sound from the gut of the person. An aspect of a state of the person determined based on the stored digital acoustic samples. Information about the determined aspect of the person's state is presented to a user.

In implementations of this and other aspects, one or a combination of two or more of the following features may be present. The sound from the gut of the person is sensed through the skin of the abdomen of the person. The predicting includes applying information about the digital acoustic samples to a predictive Bayesian model. The aspect of the state of the person includes a gut state or an emotional state. The presenting of the information to the user includes presenting a visualization associated with features of the acoustic samples. The presenting of the information to the user includes presenting audiovisual representations of the determined aspect of the person state. The aspect of the state of the person is diagnosed. A therapy for the aspect of the state of the person is identified. A prognosis for the aspect of the state of the person is provided. And experience of the person associated with the digital acoustic samples sampled.

In general, in an aspect, a device is configured to sense sound from a gut of a person and to store digital acoustic samples corresponding to the gut sound. A processor is configured to determine an aspect of a state of the person based on the stored digital acoustic samples. A device is configured to feed back representations of the determined aspect of the person's state to the person.

In implementations of this and other aspects, one or a combination of two or more of the following features may be present. The representations are configured to enable the person to understand or control the person's gut state or contributors to the gut state. The representations include audiovisual abstractions. The audiovisual abstractions include sonifications or visualizations corresponding to the state of the person. The device is configured to be worn by the person continuously and the digital acoustic samples are stored continuously. There is a device configured to sense a physical state of the person or of the person's environment. The aspect of the state of the person is based also on the physical state of the person or of the person's environment. The fed back representations include didactic or aestheticized audiovisual elements. The representations fed back to the person include diagnoses, prognoses, or therapies.

In general, in an aspect, sound is sensed from a gut of a person and digital acoustic samples corresponding to the gut sound are stored. An aspect of a state of the person is determined based on the stored digital acoustic samples. Representations of the determined aspect of the person's state are fed back to the person.

In implementations of this and other aspects, one or a combination of two or more of the following features may be present. The representations are configured to enable the person to understand or control the person's gut state or contributors to the gut state. The representations include audiovisual abstractions. The audiovisual abstractions include sonifications or visualizations corresponding to the state of the person. The device is worn by the person continuously and the digital acoustic samples are stored continuously. A physical state of the person or of the person's environment is sensed. The aspect of the state of the person is based also on the physical state of the person or of the person's environment. The fed back representations include didactic or aestheticized audiovisual elements. The representations fed back to the person include diagnoses, prognoses, or therapies

in general, in an aspect, a device is configured to sense sound from a gut of a person and to store digital acoustic samples corresponding to the gut sound. A device is configured to receive information representing an experience of the person corresponding to the gut sound. A processor is configured to determine an aspect of a state of the person based on the stored digital acoustic samples and to determine a correlation between the aspect of the state of the person and the experience of the person. A device is configured to present information about the determined aspect of the person's state and the determined correlation to a user.

In general, in an aspect, sound is sensed from a gut of a person and digital acoustic samples are stored corresponding to the gut sound. Information is received representing an experience of the person corresponding to the gut sound. An aspect of a state of the person is determined based on the stored digital acoustic samples and a correlation is determined between the aspect of the state of the person and the experience of the person information is presented about the determined aspect of the person's state and the determined correlation to a user

in general, in an aspect, a central server is configured to receive, from devices of a population of users, data representing gut sounds of each of the users and experiences of each of the users associated with the user's gut sounds. A processor is configured to analyze the gut sounds and experiences of the population and to output inferences about correlations between the gut sounds and the experiences.

In implementations of this and other aspects, one or a combination of two or more of the following features may be present. The experiences include self-assessed experiences. The experiences include one or more of thoughts, feelings, emotional states, or activities. The processor is configured to apply classification or regression to identify relationships between the experiences and gut states represented by the gut sounds. The processor is configured to output suggestions about how to understand or control gut state. The processor is configured to output predictions about future states of a person.

In general, in an aspect, data is received at a central server from devices of a population of users. The data represents gut sounds of each of the users and experiences of each of the users associated with the user's gut sounds. The gut sounds and experiences of the population are analyzed and inferences are output about correlations between the gut sounds and the experiences. The experiences include self-assessed experiences.

In implementations of this and other aspects, one or a combination of two or more of the following features may be present. The experiences include one or more of thoughts, feelings, emotional states, or activities. Classification or regression is applied to identify relationships between the experiences and gut states represented by the gut sounds. Suggestions are output about how to understand or control gut state. Predictions are output about future states of a person.

These and other aspects, features, and implementations can be expressed as methods, apparatus, systems, components, program products, methods of doing business, means or steps for performing a function, and in other ways.

These and other aspects, features, and implementations will become apparent from the following descriptions, including the claims.

### DETAILED DESCRIPTION

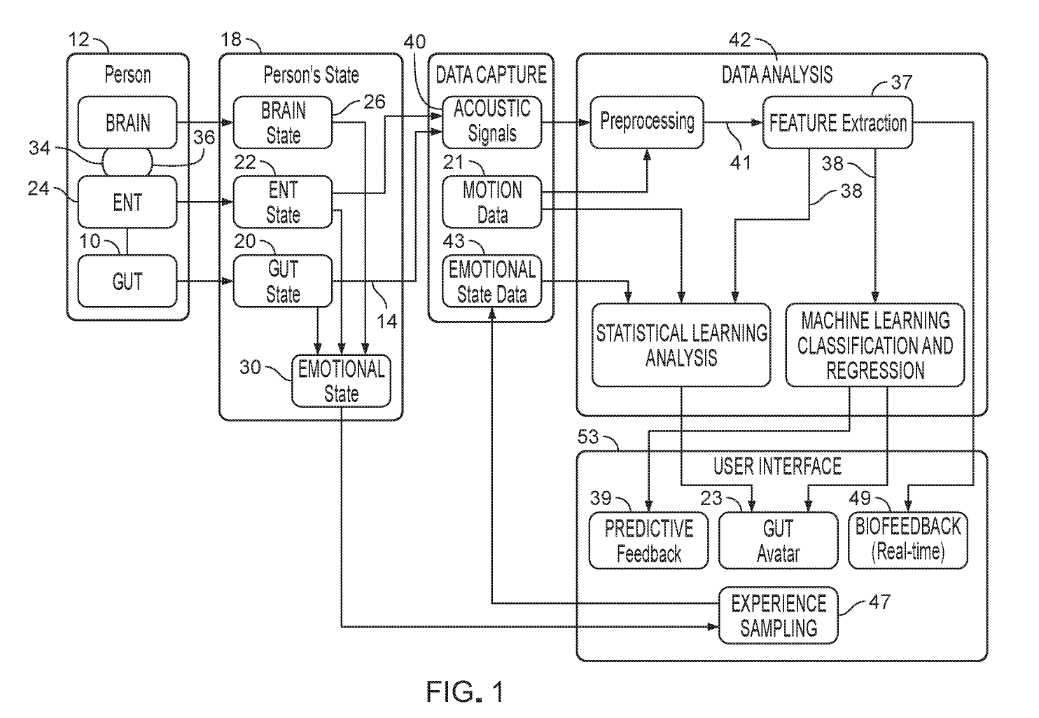

FIG. 1 is a block diagram of sound capture and use.

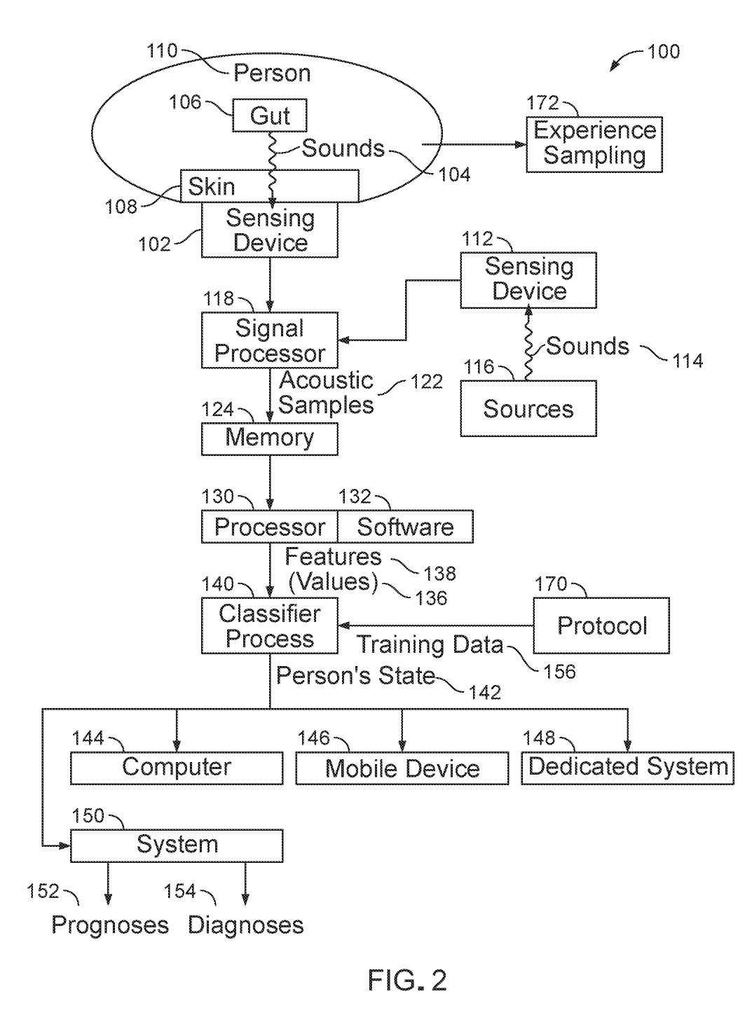

FIG. 2 is a block diagram of system for sound capture and use.

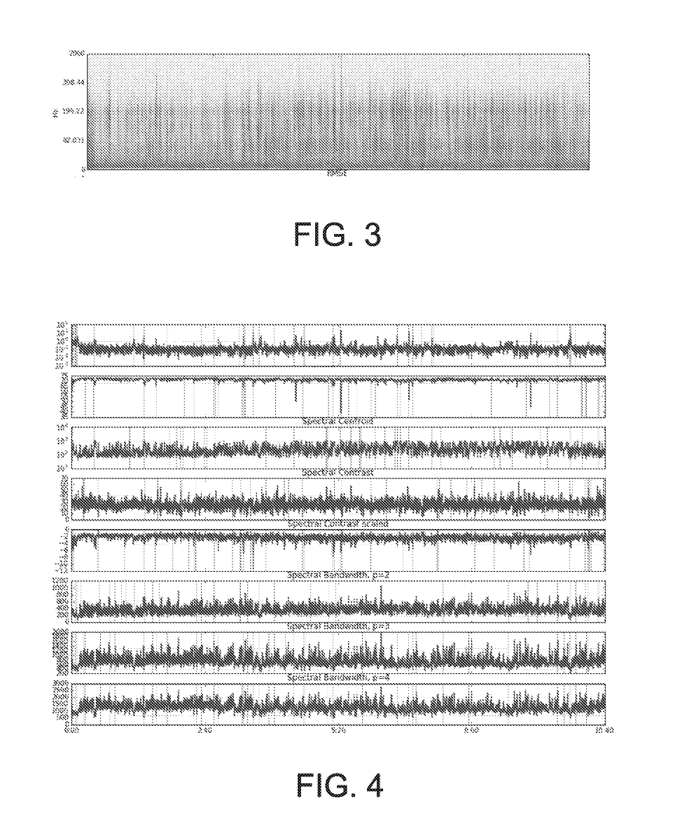

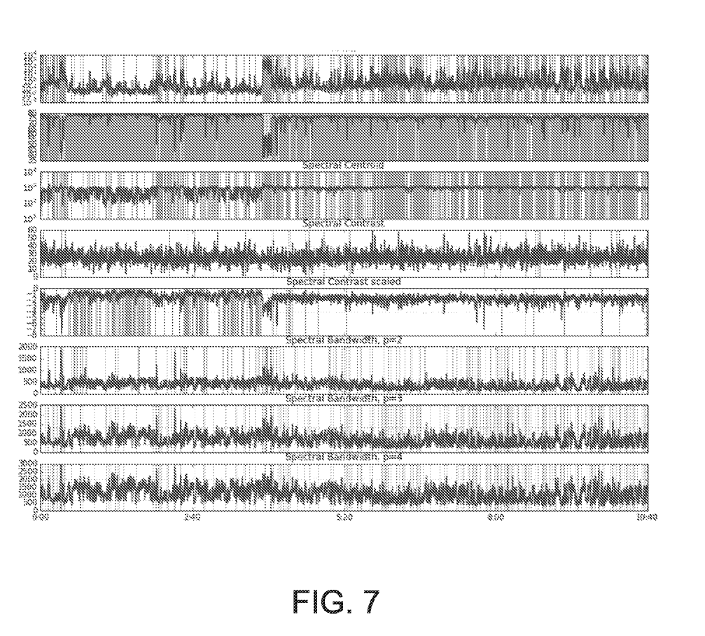

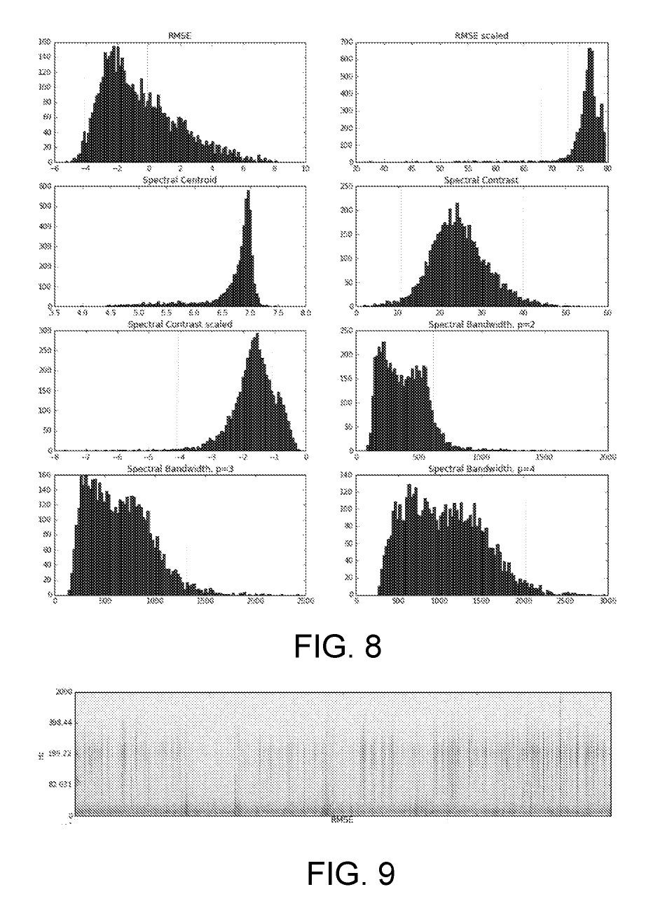

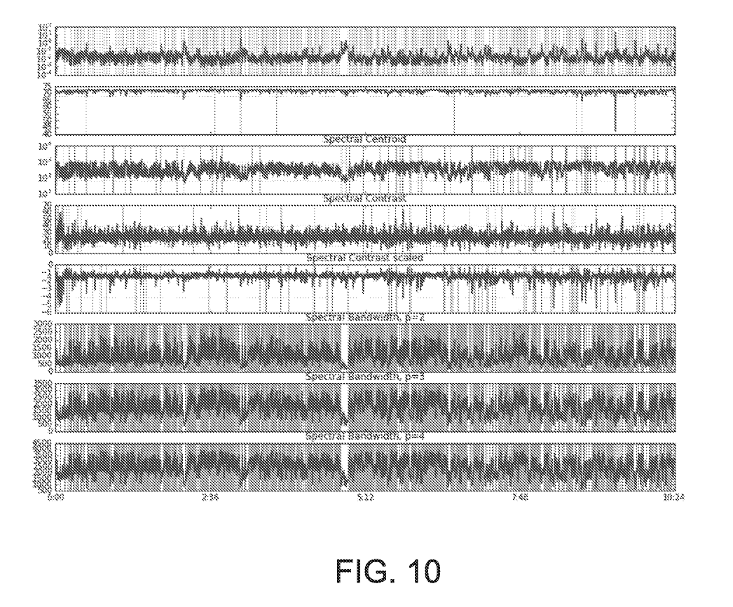

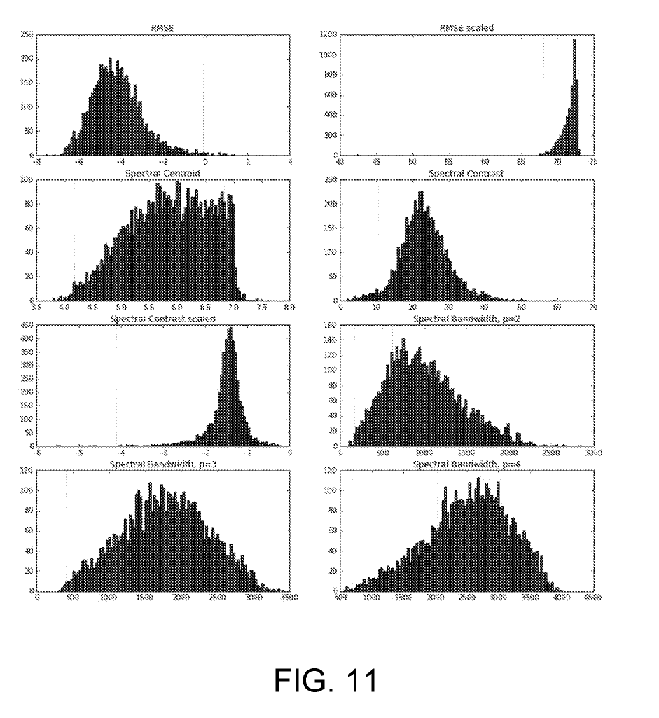

FIGS. 3 through 21, 26, and 27 show graphs.

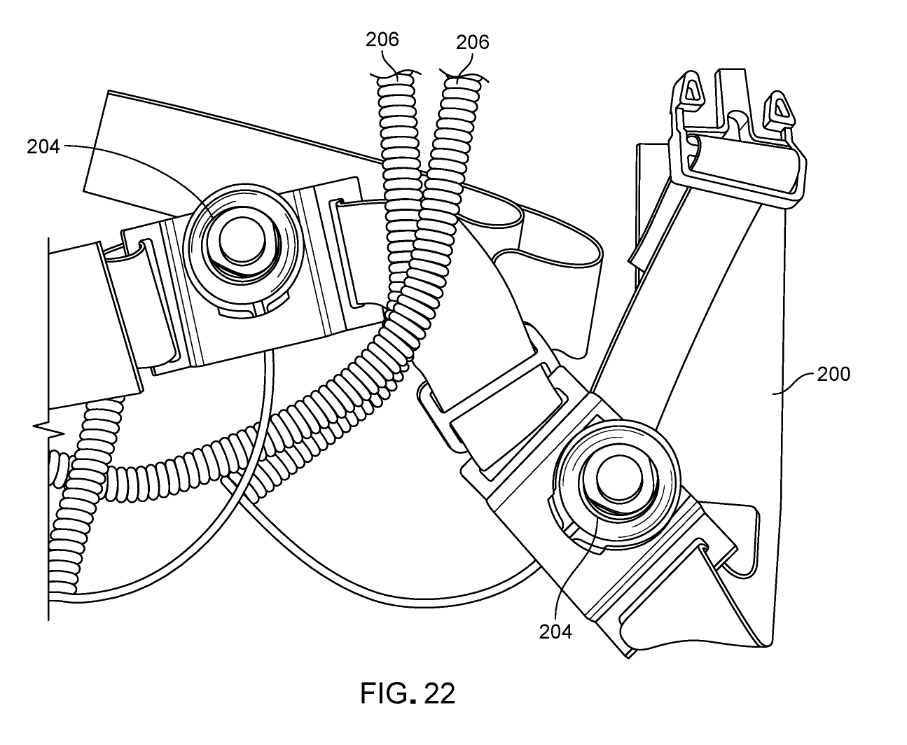

FIGS. 22 through 25 show wearable devices.

As shown in FIG. 1, the gut 10 of a person 12 produces sounds 14 that vary over time and depend on and are indicative of (a) activities of the person, and (b) past, present, and future aspects 16 of the person's state 18. The person's state may include (1) the physiological state 20 of the person's gut (the “gut state”), (2) the neurological state 22 of the person's ENT 24, (3) the neurological state 26 of the person's brain 28, (4) the emotional state 30 of the person 12, and (5) the relationships between the emotional state and the gut state and between the neurological state of the brain and neurological state of the ENT, as mediated, for example, by the vagus nerve 34 and other pathways 36.

Acoustic signals 40 can be derived by sensing the gut sounds over time. Certain features 38 of the acoustic signals can then be correlated with the past, present, and future aspects of the person's state and the information can be used for a wide variety of purposes including (a) in general, characterizing, understanding, inferring, predicting, or altering aspects of the person's state, or the relationships between the gut state and the emotional state and between the ENT state and the brain state, (b) extracting descriptive features of the acoustic signals and studying the features, (c) training and using a classifier or other machine learning system 42, (d) predicting a future state of the person including a future gut state and a future emotional state, (e) inferring aspects of the past state of the person, including gut state or emotional state, (f) determining aspects of the current state of the person such as gut state and emotional state, (g) changing aspects of the person's current or future state including the current or future gut state and emotional state, (h) providing bio-feedback to the person, (i) presenting information through a user interface for research, study, and other purposes, (j) accumulating data about aspects of the states of individual people or populations, including gut states and emotional states, and relationships of states for training and study purposes, and combinations of (a) through (j), among others. The data analysis engine 42 also can receive, preprocess, and use in feature extraction, motion data 21 that represents motion of the user, for example, contemporaneous with the acoustic samples.

Here we describe technologies that include systems, devices, and techniques useful for (a) sensing gastric sounds over time, (b) generating corresponding acoustic signals and storing the acoustic signals as digital samples (acoustic samples 41), (c) processing the acoustic samples to characterize, understand, predict, or alter the gut state, the emotional state or other aspects of the state of the person, or the relationship between the gut state and the emotional state or the ENT state and the brain state; (d) providing the information 43 about aspects of the state of the person, to the person, in some cases for real-time and continuous bio-feedback 49; (e) developing, storing, and providing training data and feature sets for use in machine learning systems; (f) combining information about the person's state 43 with experience sampling 47 received from the person; (g) predicting and providing predictive feedback 39 on aspects of a person's state, such as gut state or emotional state; (h) presenting user interfaces 53 including predictive feedback, real time bio-feedback, and gut avatar 23 interfaces, and (i) providing accumulative (in addition to real-time) bio-feedback in the form of an evolving visualization that reflects a person's past, present, or future state in a (highly) stylized and personalized visual form (e.g., using an avatar), among other things.

In considering the relationships of gut state to emotional state or ENT state to brain state, the manner in which the gut state or ENT state connects to or affects the emotional state or the brain state is of particular interest for the technologies that we describe here.

In our discussion, we use the terms “gastric,” “gut,” and “stomach” broadly to include, for example, any part or parts of the gastrointestinal tract and related organs of a person that produce sounds that can be perceived externally to the body, including, for example, the stomach, the small intestine, and the large intestine.

Among the markets of interest for products and implementations of the systems, devices, and techniques that we describe here are health care providers, researchers, device makers, consumers, sports trainers, and psychotherapists, and others.

As shown in FIG. 2, some implementations of a technology 100 useful for the purposes and in the ways discussed above can include the following components, among others.

A sensing device 102 (or an array of two or more devices) senses sounds 104 that are produced by the gut 106 and propagate through the skin 108 of a person 110. In some implementations, a second sensing device 112 (which can be placed at some distance from the gut) senses sounds 114 produced by sound sources 116 (e.g., speech of the person or sounds from ambient sources) that may represent noise with respect to the sensed gut sounds. A signal processor 118 digitizes the sensed gut sounds and the sensed other sounds 114 and processes them to produce a stream (time series) of digital signal samples (acoustic samples) 122 representing the gut sounds. The signal samples 122 are stored in a flash or other memory 124. Sensing devices 125 capture information about the person's physical movements and or other environmental factors including time of day, temperature, atmospheric pressure, humidity, or ambient noise level. The captured information can be provided to the classifier process to be taken into account in classifying a person's state.

A processor 130 (under control of software 132) immediately or at a later time fetches from the memory 124 and processes the acoustic samples belonging to one or more segments (or all) of the stream to derive values 136 for features 138 that are known to have (or may be determined to have) a correlation to one or more aspects of the person's state, including the gut state and the emotional state, for example. In some implementations, one of the first steps of the processing is to determine the spectral characteristics of the acoustic samples.

The processor 130 can be configured to apply the feature values to a classifier process 140 to train the classifier process or (once trained) to use the classifier process to classify the features (associated with newly sensed gut sounds) and produce a classifier output 142 that is, for example, indicative of aspects of the person's state, such as the gut state or the emotional state, among others.

The classifier output 142 can be used for a wide variety of purposes and in a wide variety of applications.

In some cases, the classifier output 142 can be used by software running on a computer 144 or mobile device 146 to enable, for example, a user to study the features and feature values and their correspondence to specific aspects of the person's state.

In some instances, the classifier output 142 can be used by software running on or in connection with a dedicated (for example, wearable) system 148 to provide feedback (e.g., bio-feedback) to a user to enable the user to understand aspects of her state, including her gut state and her emotional state, or to consciously or subconsciously alter the behavior of her gut, ENT, vagus nerve, or brain to improve or otherwise change her state. In some situations, the feedback can predict a future state, such as a statement to a user that “You may have a stomach ache in 15 minutes.”

In some implementations, the classifier output 142 can be used by software running on a system 150 that provides diagnoses 152 of subjects, prognoses 154, or suggestions of useful therapies to correct or improve aspects of the state of subjects, including gut state and emotional state. For applications of this kind, we use training samples from known states (acquired, for example, in an experimental stimulus setting), to estimate a distribution of features of each state, and then classify future segments of time based on their posterior probability over states based on the estimated distributions.

For purposes of determining the features that correlate well to aspects of a person's state and therefore can be used to configure the classifier process 140, the classifier processor can be trained based on sets of training data. A set of training data 156 could include, for example, a stream of acoustic samples taken from a subject under known conditions (say, one hour after eating) or who has known aspects 160 of her state, such as a person known to have IBD.

For the purpose of creating a set of training data during the training process or a set of “real” data once the training has been completed, a protocol 170 (described later) can be established that defines an overall time period for the accumulation of a stream of signal samples, segments within that overall time period, for example, a segment that covers a one-hour period immediately after eating, and other controlled contextual factors.

In some implementations, in addition to capturing acoustic samples of gut sounds, experiences of the user can be sampled, for example, by asking the user for data (experience sampling) 172 about the user's past and current states, including gut states, emotional states, activities and other persons they are interacting with. This can be done simultaneously during the capturing of the acoustic samples, or at another time. The experience data can be used during the capturing of training data or during the operation of the classifier at “run” time. Experience sampling can be used for a wide variety of purposes including, in combination with the information about a person's state 142 generated by the classifier process, to alter or improve determinations about the person's state.

In some cases, the second sensing device 112 can be used to identify segments of time in the recording of gut sounds in which naively interesting feature shifts are actually caused by ambient (e.g., room) noise. The identification procedure can incorporate one or more of (a) standard noise cancellation techniques from audio analysis, (b) shifts in the distributions of features of interest in the ambient noise recording, or (c) a separately-trained predictive model of gut feature shifts based on ambient recording features.

Removing gut sound segments corrupted by background noise can be done during the training and classification procedures. Excluded segments are not included in the stimulus model estimation or in the posterior computation. The classification model is able to accommodate these deletions of corrupted portions of data.

The simple scheme (b) for removing noisy blocks can be represented as follows:

[Block of background sound data]->[Feature extraction]->[Evaluation for particularly high signal under any stimulus model]->[Exclude this block]

The complex (supervised) scheme (c) for excluding noisy blocks can be represented this way:

Training Background Filter:

[Block of background sound data], [State of gut signal during this time]->[Feature extraction]->[Model to predict abnormal features in gut signal]

Evaluating Background Filter:

[Block of background sound data]->[Feature extraction]->[Predictions of gut signal distortion]->[Exclude this block]

Example of Classifier Process

Here we describe and show Python code and resulting visualizations associated with an example classifier process.

Loading Code and Data

As shown below, the code and then the data are loaded. The code is in two parts, one part is for loading data, generating features (e.g., spectral features) from the data, and plotting the data and features for analysis (e.g., producing visualizations of the data); the other part is for modeling and evaluating feature distributions and carrying out nonparametric naïve Bayesian classification on the features.

The data in this example includes a set of data derived from a gut state of an empty stomach (in this case about 10 minutes of data). Spectral information about the empty stomach data is computed. Then about 10 minutes of data for a gut state of eating is loaded and spectrally processed, followed by loading data for an emotional state of disgust.

A selected set of features are then computed for each of the three data sets.

| | |
|---|---|
| In [1]: | import sys |
| In [2]: | #Import the code for loading data, generating features, an |
| | d plotting data and features |
| | %run code/data_load_and_plot.py |
| In [3]: | #Import the code for modeling and evaluating feature distr |
| | ibutions and carrying out nonparametric naive Bayes |
| | %run code/kde_models.py |
| In [4]: | #Load the empty stomach data (first 10 min or so) |
| | data_empty, sr_empty = sf.read(“./data/george_empty.WAV”, |
| | stop = 1536*40000) |
| | #Precompute the spectrogram and feed it to the plotting fu |
| | nction for speed |
| | spec_empty = get_spectrogram(data_empty, sr_empty, 4000) |
| In [5]: | #Load the eating data (first 10 min or so) |
| | data_eat, sr_eat = sf.read(“./data/george_full.WAV”, |
| | stop = 1536*40000) |
| | spec_eat = get_spectrogram(data_eat, sr_eat, 4000) |
| In [6]: | #Load the disgust data. |
| | data_disgust, sr_disgust = sf.read(“./data/george_disgust. |
| | WAV”, stop = 1536*40000) |
| | spec_disgust = get_spectrogram(data_disgust, sr_disgust, 4 |
| | 000) |
| In [7]: | #Compute all our features |
| | features_empty = get_features(data_empty, spec_empty) |
| | features_eat = get_features(data_eat, spec_eat) |
| | features_disgust = get_features(data_disgust, spec_disgust |
| | ) |

Visualizations

Next, as illustrated by the code example below, the system generates data and feature visualizations.

FIG. 3 is a resulting spectrogram visualization for a set of data acquired from an empty stomach, with the signal power plotted versus frequency (y-axis) and time (x-axis). Darker regions correspond to higher power. The features extracted in this example are functions of this spectrogram.

FIG. 4 shows visualizations of values over time of each of eight different features extracted from feature sets identified by running the code on the input data set for an empty stomach. For this example, standard features are computed using the librosa [https://librosa.github.io/] software package. Each feature is a function of the spectrogram from FIG. 3, measured for each time bin: the overall power under two different normalizations, the central frequency of the power distribution (spectral centroid), the spectral contrast under two normalizations, and the standard deviation, skewness, and kurtosis of the frequency distribution (spectral bandwidth p=2,3,4). Regions of extreme high or low values (below 2.5% or above 97.5% quantiles) are shaded, dark gray for high values and light gray for low values.

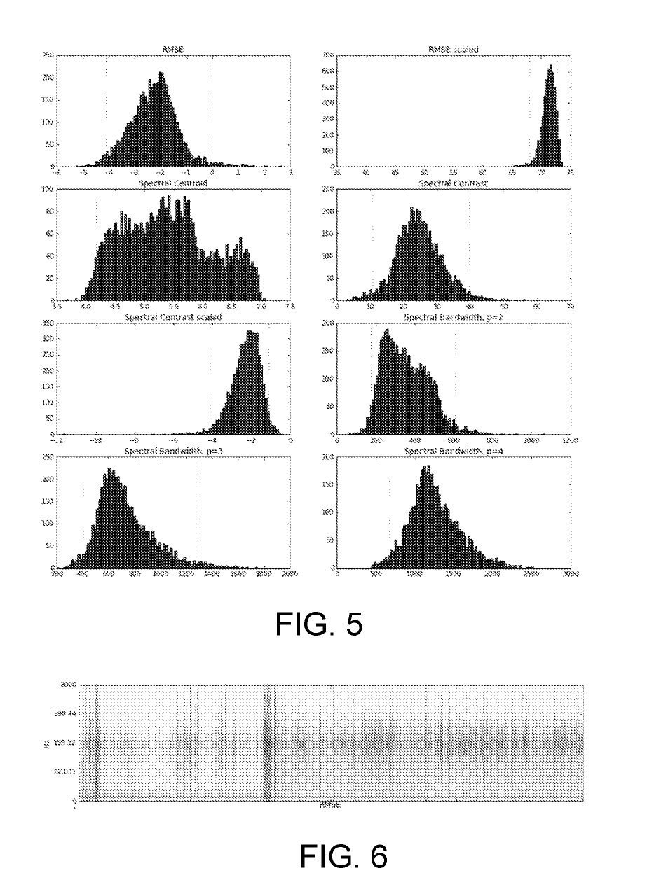

FIG. 5 shows visualizations (histograms) of the marginal distributions of each feature over the ten-minute recording, showing the feature value (x-axis) and the number of time bins with that feature value range (y-axis). The dotted vertical lines show the 2.5% and 97.5% quantiles for each feature.

FIGS. 6, 7, and 8, and FIGS. 9, 10, and 11, are visualizations analogous to FIGS. 1, 2, and 3, for the gut state after eating and for the emotional state of disgust, respectively. The dotted lines show the 2.5% and 97.5% quantiles of the corresponding empty stomach feature distributions, demonstrating a shift in the feature distributions in the after eating and disgust states.

In FIGS. 3, 6, 9, the points where each feature is higher or lower than the 2.5% highest or lowest feature values in the gut state of an empty stomach are highlighted respectively. This may make shifts in distributions easier to see. Our later models will incorporate these shifts in distribution for classification.

| | |
|---|---|
| In [8]: | #Plot empty stomach spectrogram and features |
| | plotclip(data_empty, sr_empty, ‘Ali: Empty Stomach’, feat |
| | ures = features_empty, spec=spec_empty, |

cutoffs=features_empty[‘cutoffs’])

| | |
|---|---|
| In [9]: | #Plot after eating spectrogram and features, using empty |
| | stomach cutoffs |
| | plotclip(data_eat, sr_eat, ‘Ali: After Eating’, |
| | features = features_eat, spec=spec_eat, |

cutoffs=features_empty[‘cutoffs’])

| | |
|---|---|
| In [10]: | #Plot disgust spectrogram and features, using empty stoma |
| | ch cutoffs |
| | plotclip(data_disgust, sr_disgust, ‘Ali: Disgust’, spec=s |
| | pec_disgust, features = features_disgust, |

cutoffs=features_empty[‘cutoffs’])

Probabilistic Modeling

As illustrated in the code below, the system applies a model to the eight features described above.

As a preliminary model, the system uses a naive Bayes model with nonparametric estimation of the univariate feature distributions within class. The get kernel function fits kernel density estimates to each feature. The eval kernel logprobs function evaluates the log probabilities for the observed features from a new set of data against one of these sets of density estimates.

Initially the system splits each of the three data sets in half so that the model can be trained on one-half of each data set and later validated using the other half of the data set.

Next the system computes univariate kernel densities for each feature and produces visualizations.

| | |
|---|---|
| In [11]: | #Sample splitting, so we can train on one half and validat |
| | e on the other |
| | tmp = split_feature(features_empty) |
| | features_empty1 = tmp[‘features1’]#Train |
| | features_empty2 = tmp[‘features2’]#Test |
| | tmp = split_feature(features_eat) |
| | features_eat1 = tmp[‘features1’] |
| | features_eat2 = tmp[‘features2’] |
| | tmp = split_feature(features_disgust) |
| | features_disgust1 = tmp[‘features1’] |
| | features_disgust2 = tmp[‘features2’] |
| In [12]: | #Compute univariate kernel densities for each feature |
| | kernels_empty = get_kernels(features_empty1) |
| | kernels_eat = get_kernels(features_eat1) |
| | kernels_disgust = get_kernels(features_disgust1) |
| In [13]: | plot_features_with_kernels(features_empty, kernels_empty) |
| In [14]: | compare_kernels([kernels_empty,kernels_eat,kernels_disgust |
| | ],[features_empty,features_eat,features_disgust]) |

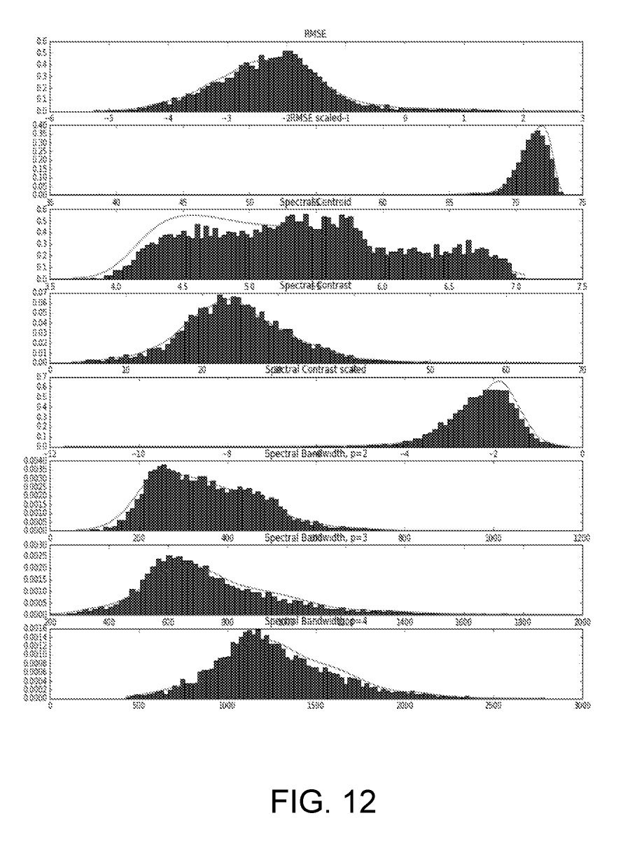

The univariate distributions for each class by feature give a sense for how the models work. First, plots of the estimated distributions for each feature in the empty stomach data (FIG. 12) are overlaid on histograms to show how the models approximate the feature histograms (FIGS. 5, 8, and 11).

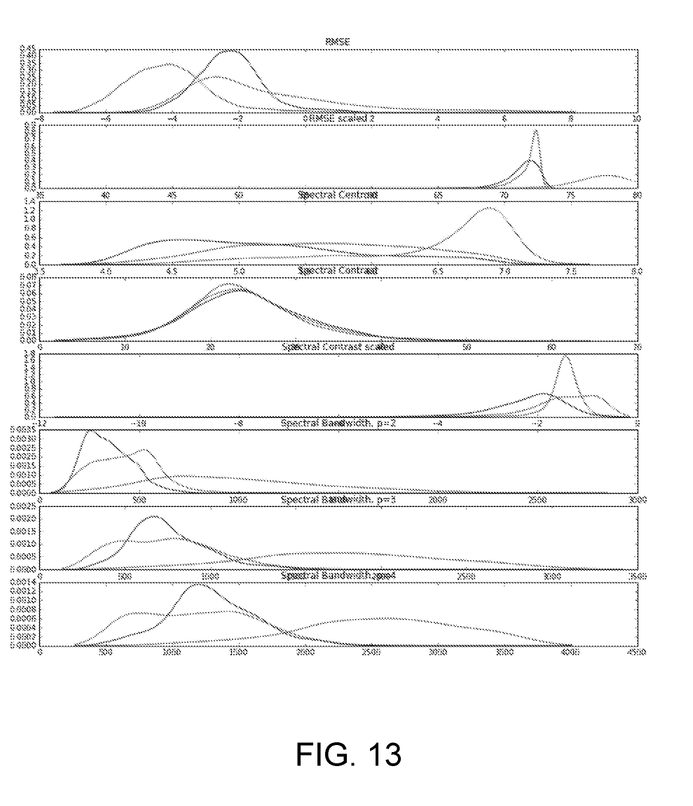

Next, the feature models for each condition (state) can be compared. In FIG. 13, the lines represent estimated densities (corresponding to histograms in FIGS. 5, 8, and 11) for each feature under each of the three states. Non-overlap in the distributions between the conditions (states) can be leveraged for classification. In the plots shown, the estimated feature distributions for the disgust state and eating state, for example, are much closer to each other than they are to the empty stomach state.

Using the Probabilistic Models to Build a Simple Classifier

As illustrated by the code shown below, the feature models formed in the steps described above can be used to form a simple naive Bayes model of the posterior class distribution given a time sequence of acoustic samples. For each time point, the system computes the log probability of the observed feature under each non-parametrically estimated state distribution (In[15] in the code). Under the model that both time points and features are independent, the sum of these terms over time and features would give the classification score (under equal class priors). Note that this independence model does not need to hold true in reality for the classifier to work well in practice.

The other portions of the code are described following the code.

| | |
|---|---|
| In [15]: | #Evaluate the probability of the empty timeseries using e |
| | ach kernel |
| | logprob_empty_as_empty = eval_kernel_logprobs(features_em |
| | pty2, kernels_empty) |
| | logprob_empty_as_eat = eval_kernel_logprobs(features_empt |
| | y2, kernels_eat) |
| | logprob_empty_as_disgust = eval_kernel_logprobs(features_{—} |
| | empty2, kernels_disgust) |
| | #Evaluate the probability of the after eating timeseries |
| | using each kernel |
| | logprob_eat_as_empty = eval_kernel_logprobs(features_eat2 |
| | , kernels_empty) |
| | logprob_eat_as_ eat = eval_kernel_logprobs(features_eat2, |
| | kernels_eat) |
| | logprob_eat_as_disgust = eval_kernel_logprobs(features_ea |
| | t2, kernels_disgust) |
| | #Evaluate the probability of the disgust timeseries using |

each kernel

| | |
|---|---|
| | logprob_disgust_as_empty = eval_kernel_logprobs(features_{—} |
| | disgust2, kernels_empty) |
| | logprob_disgust_as_eat = eval_kernel_logprobs(features_di |
| | sgust2, kernels_eat) |
| | logprob_disgust_as_disgust = eval_kernel_logprobs(feature |
| | s_disgust2, kernels_disgust) |
| In [16]: | fig, axes = plt.subplots(nrows = len(logprob_eat_as_empty |
| | ), ncols=1, sharex=False, sharey=False,figsize=(14, 14)) |
| | fig.suptitle(‘Instantaneous class scores for empty stomac |
| | h data’, size=20) |
| | for i in range(len(logprob_eat_as_empty)): |

plt.subplot(axes[i])
plt.plot(logprob_empty_as_eat[i],alpha=0.6, label=‘Afte

r eating score’)

plt.plot(logprob_empty_as_empty[i],alpha=0.6, label=‘Em

pty stomach score’)

title = features_empty[‘feat_titles’][i]
plt.title(title)

| | |
|---|---|
| | plt.legend(bbox_to_anchor=(0, 10), loc=2, borderaxespad=0 |
| | .) |
| | plt.show( ) |
| In [17]: | fig, axes = plt.subplots(nrows = len(logprob_eat_as_empty |
| | ), ncols=1, sharex=False, sharey=False,figsize=(14, 14)) |
| | fig.suptitle(‘Instantaneous class scores for after eating |

data’, size=20)

for i in range(len(logprob_eat_as_empty)):

plt.subplot(axes[i])
plt.plot(logprob_eat_as_eat[i],alpha=0.6, label=‘After

eating score’)

plt.plot(logprob_eat_as_empty[i],alpha=0.6, label=‘Empt

y stomach score’)

title = features_empty[‘feat_titles’][i]
plt.title(title)

| | |
|---|---|
| | plt.legend(bbox_to_anchor=(0, 10), loc=2, borderaxespad=0 |
| | .) |
| | plt.show( ) |
| In [18]: | fig, axes = plt.subplots(nrows = len(logprob_empty_as_emp |
| | ty), ncols=1, sharex=False, sharey=False,figsize=(14, 14) |
| | ) |
| | for i in range(len(logprob_empty_as_empty)): |

plt.subplot(axes[i])
plt.plot(np.cumsum(logprob_empty_as_eat[i]−logprob_empt

y_as_empty[i]))

cert = 0.99
plt.axhline(y=np.log(cert/(1−cert)), color=‘grey’, line

style=‘dotted’,alpha=0.5)

plt.axhline(y=np.log(((1−cert)/(cert))), color=‘grey’,

linestyle=‘dotted’,alpha=0.5)

title = features_empty[‘feat_titles’][i]
plt.title(title)

fig.suptitle(‘Cumulative class scores comparing eaten vs.

empty for empty stomach data’, size=20)

| | |
|---|---|
| | plt.show( ) |
| In [19]: | fig, axes = plt.subplots(nrows = len(logprob_empty_as_emp |
| | ty), ncols=1, sharex=False, sharey=False,figsize=(14, 14) |
| | ) |
| | for i in range(len(logprob_empty_as_empty)): |

plt.subplot(axes[i])
plt.plot(np.cumsum(logprob_eat_as_eat[i]−logprob_eat_as

_empty[i]))

cert = 0.99
plt.axhline(y=np.log(cert/(1−cert)), color=‘grey’, line

style=‘dotted’,alpha=0.5)

plt.axhline(y=np.log(((1−cert)/(cert))), color=‘grey’,

linestyle=‘dotted’,alpha=0.5)

title = features_empty[‘feat_titles’][i]
plt.title(title)

fig.suptitle(‘Cumulative class scores comparing eaten vs.

empty for after eating data’, size=20)

| | |
|---|---|
| | plt.show( ) |
| In [20]: | fig, axes = plt.subplots(nrows = len(logprob_eat_as_empty |
| | ), ncols=1, sharex=False, sharey=False,figsize=(14, 14)) |
| | for i in range(len(logprob_eat_as_empty)): |

plt.subplot(axes[i])
plt.plot(np.cumsum(logprob_eat_as_disgust[i]−logprob_ea

t_as_eat[i]))

cert = 0.99
plt.axhline(y=np.log(cert/(1−cert)), color=‘grey’, line

style=‘dotted’,alpha=0.5)

plt.axhline(y=np.log(((1−cert)/(cert))), color=‘grey’,

linestyle=‘dotted’,alpha=0.5)

plt.title(features_empty[‘feat_titles’][i])

| | |
|---|---|
| | fig.suptitle(‘Cumulative class scores comparing disgust v |
| | s. eaten for after eating data’, size=20) |
| | plt.show( ) |
| In [21]: | fig, axes = plt.subplots(nrows = len(logprob_eat_as_empty |
| | ), ncols=1, sharex=False, sharey=False,figsize=(14, 14)) |
| | for i in range(len(logprob_eat_as_empty)): |

plt.subplot(axes[i])
plt.plot(np.cumsum(logprob_disgust_as_disgust[i]−logpro

b_disgust_as_eat[i]))

cert = 0.99
plt.axhline(y=np.log(cert/(1−cert)), color=‘grey’, line

style=‘dotted’,alpha=0.5)

plt.axhline(y=np.log(((1−cert)/(cert))), color=‘grey’,

linestyle=‘dotted’,alpha=0.5)

plt.title(features_empty[‘feat_titles’][i])

| | |
|---|---|
| | fig.suptitle(‘Cumulative class scores comparing disgust v |
| | s. eaten for disgust data’, size=20) |
| | plt.show( ) |
| In [22]: | fig, axes = plt.subplots(nrows = 2, ncols=2, sharex=False |
| | , sharey=False,figsize=(14, 10)) |
| | alpha = 0.6 |
| | plt.subplot(axes[0,0]) |
| | plt.plot(sum(logprob_empty_as_empty), alpha=alpha, label= |
| | ‘Empty score’) |
| | plt.plot(sum(logprob_empty_as_eat), alpha=alpha, label=‘A |
| | fter eating score’) |
| | plt.title(‘Empty stomach sample’) |
| | plt.legend( ) |
| | plt.subplot(axes[0,1]) |
| | plt.plot(sum(logprob_eat_as_empty), alpha=alpha, label=‘E |
| | mpty score’) |
| | plt.plot(sum(logprob_eat_as_eat), alpha=alpha, label=‘Aft |
| | er eating score’) |
| | plt.title(‘After eating sample’) |
| | #plt.legend(bbox_to_anchor=(0, 0) , loc=2, borderaxespad=0 |
| | .) |
| | plt.legend( ) |
| | plt.subplot(axes[1,0]) |
| | plt.plot(sum(logprob_eat_as_eat), alpha=alpha, label=‘Aft |
| | er eating score’, color=‘#ff7f0e’) |
| | plt.plot(sum(logprob_eat_as_disgust), alpha=alpha, label= |
| | ‘Disgust score’, color=‘g’) |
| | plt.title(‘After eating sample’) |
| | plt.legend( ) |
| | plt.subplot(axes[1,1]) |
| | plt.plot(sum(logprob_disgust_as_eat), alpha=alpha, label= |
| | ‘After eating score’, color=‘#ff7f0e’) |
| | plt.plot(sum(logprob_disgust_as_disgust), alpha=alpha, la |
| | bel=‘Disgust score’, color=‘g’) |
| | plt.title(‘Disgust sample’) |
| | plt.legend( ) |
| | plt.suptitle(‘Instantaneous scores’,size=20) |
| | plt.show( ) |
| In [23]: | fig, axes = plt.subplots(nrows = 4, ncols=1, sharex=False |
| | , sharey=False,figsize=(10, 16)) |
| | subalpha = 0.6 |
| | #Empty vs. eaten, truth empty |
| | plt.subplot(axes[0]) |
| | plt.plot(np.cumsum(sum(logprob_empty_as_eat)−sum(logprob_{—} |
| | empty_as_empty)),label=‘Total Cumulative Score’) |
| | for i in range(len(logprob_eat_as_disgust)): |

plt.plot(np.cumsum(logprob_empty_as_eat[i]−logprob_empt

y_as_empty[i]),‘:’ ,label=features_empty[‘feat_titles’][i
])
plt.title(‘Empty vs. Eaten score. Truth: Empty’)
plt.legend(bbox_to_anchor=(1.05, 1), loc=2, borderaxespad
=0.)
#Empty vs. eaten, truth eaten
plt.subplot(axes[1])
plt.plot(np.cumsum(sum(logprob_eat_as_eat)−sum(logprob_ea
t_as_empty)),label=‘Total Cumulative Score’)
for i in range(len(logprob_eat_as_disgust)):

plt.plot(np.cumsum(logprob_eat_as_eat[i]−logprob_eat_as

_empty[i]),‘:’ ,label=features_empty[‘feat_titles’][i])
plt.title(‘Empty vs. Eaten score. Truth: Eaten’)
plt.legend(bbox_to_anchor=(1.05, 1), loc=2, borderaxespad
=0.)
#Eat as eat vs. disgust, truth eaten
plt.subplot(axes[2])
plt.plot(np.cumsum(sum(logprob_eat_as_disgust)−sum(logpro
b_eat_as_eat)),label=‘Total Cumulative Score’)
for i in range(len(logprob_eat_as_disgust)):

plt.plot(np.cumsum(logprob_eat_as_disgust[i]−logprob_ea

t_as_eat[i]),‘:’ ,label=features_empty[‘feat_titles’][i])
plt.title(‘Disgust vs. Eaten score. Truth: Eaten’)
plt.legend(bbox_to_anchor=(1.05, 1), loc=2, borderaxespad
=0.)
#Eat as eat vs. disgust, truth disgust
plt.subplot(axes[3])
plt.plot(np.cumsum(sum(logprob_disgust_as_disgust)−sum(lo
gprob_disgust_as_eat)),label=‘Total Cumulative Score’)
for i in range(len(logprob_eat_as_disgust)):

plt.plot(np.cumsum(logprob_disgust_as_disgust[i]−logpro

b_disgust_as_eat[i]),‘:’ ,label=features_empty[‘feat_titl
es’][i])
plt.title(‘Disgust vs. Eaten score. Truth: Disgust’)
plt.legend(bbox_to_anchor=(1.05, 1), loc=2, borderaxespad
=0.)
plt.show( )

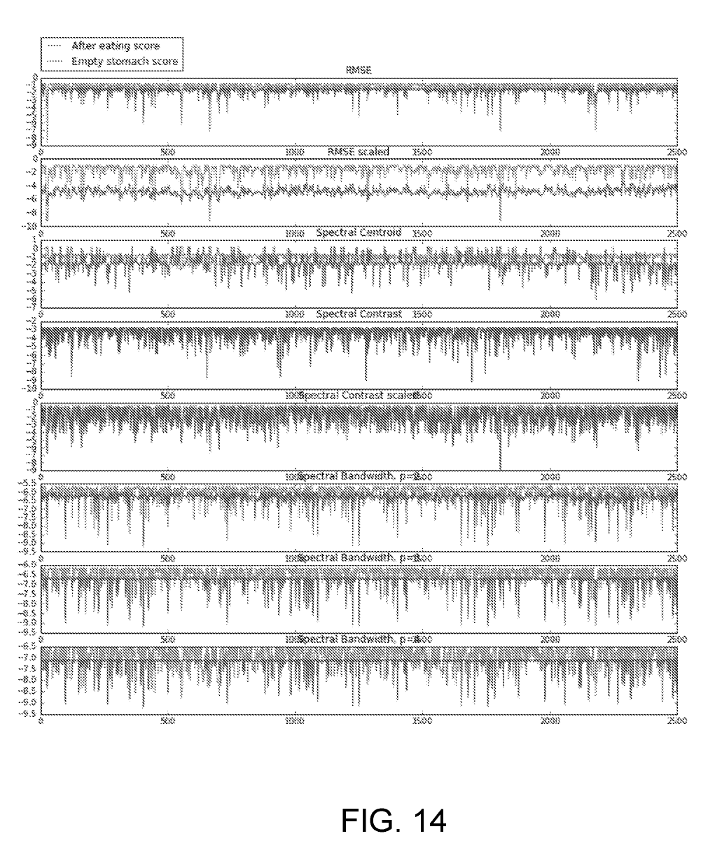

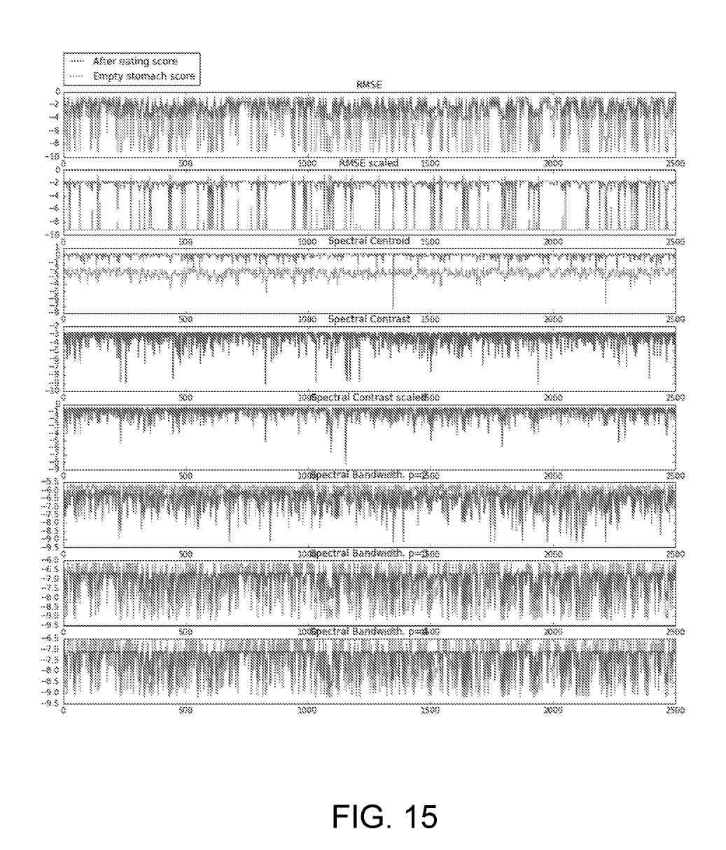

At In[16], an understanding of the models can be learned from visualizations of the score for each time point of each feature. FIG. 14 shows the visualization of the scoring using data from an empty stomach, scored for both the empty stomach distribution and the after eating distribution. The scores for empty stomach tend to be larger, indicating that this data looks more similar to the empty stomach state than the after eating state. The RMSE scaled feature is particularly informative in this example.

At In[17], the code applies the same process to data from the after eating state, scored against both classes. As shown in the visualization of the results (in FIG. 15), the after eating class tends to receive higher scores, indicating that this data looks more similar to the after eating state than the empty stomach state.

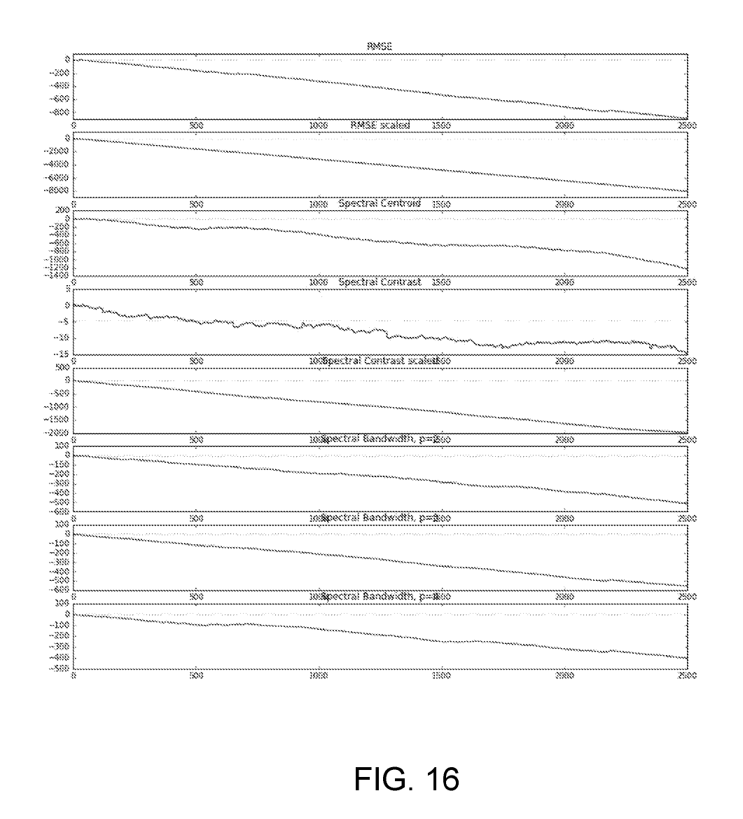

The difference in instantaneous score values (FIGS. 14 and 15) between states can be combined over time to obtain an overall classification score between states. The code at In[18] calculates and produces visualizations of the difference in cumulative sums of the scores between two classes, which indicates the accumulated discriminating information up to a given point in time. These visualizations are shown in FIG. 16.

These cumulative scores for each feature can be viewed first to get an idea of the relative information contribution of each feature. The overall classification score is the sum of the scores across features.

For a sense of scale, a cumulative score of about 5 gives a 99% posterior probability (in a balanced setting) when the independence assumptions mentioned earlier are actually true. The dependencies in our case will make this threshold larger, but it's a useful benchmark.

For the cumulative scores of eaten versus empty, for an empty stomach sample (FIG. 16), there is classification strength if the scores drop over time, because the samples are truly from an empty stomach. The scale of the y-axis for each feature gives an indication of the relative value of each feature. For classification in this example, scaled RMSE is providing greater classification strength than the other features, demonstrated by the scores at the end of the time interval being much more negative.

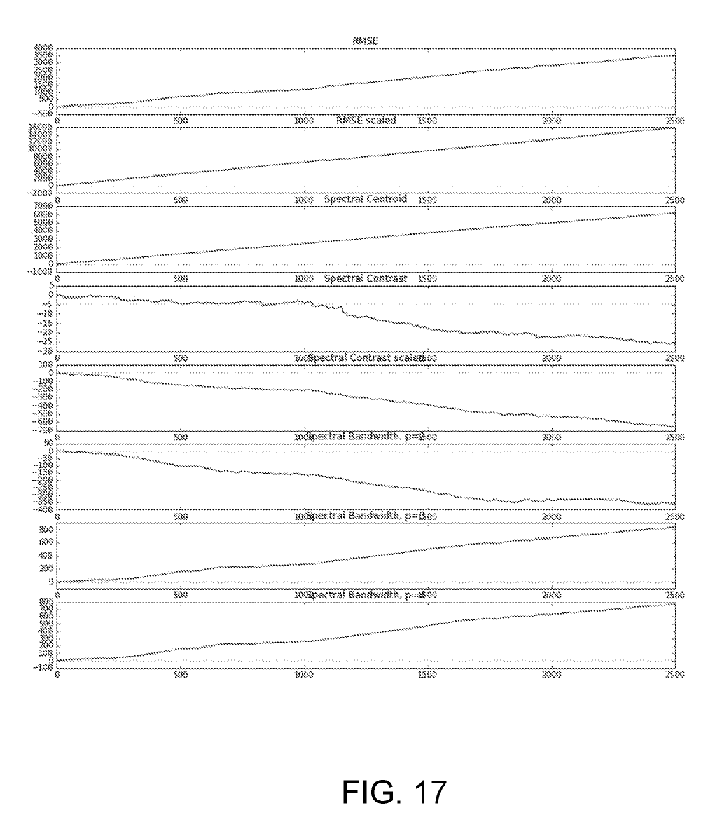

The code at In[19] executes a similar process using after eating samples. Here, there is classification strength if the scores rise over time. As shown in FIG. 17, the scores rise for some features and drop for others.

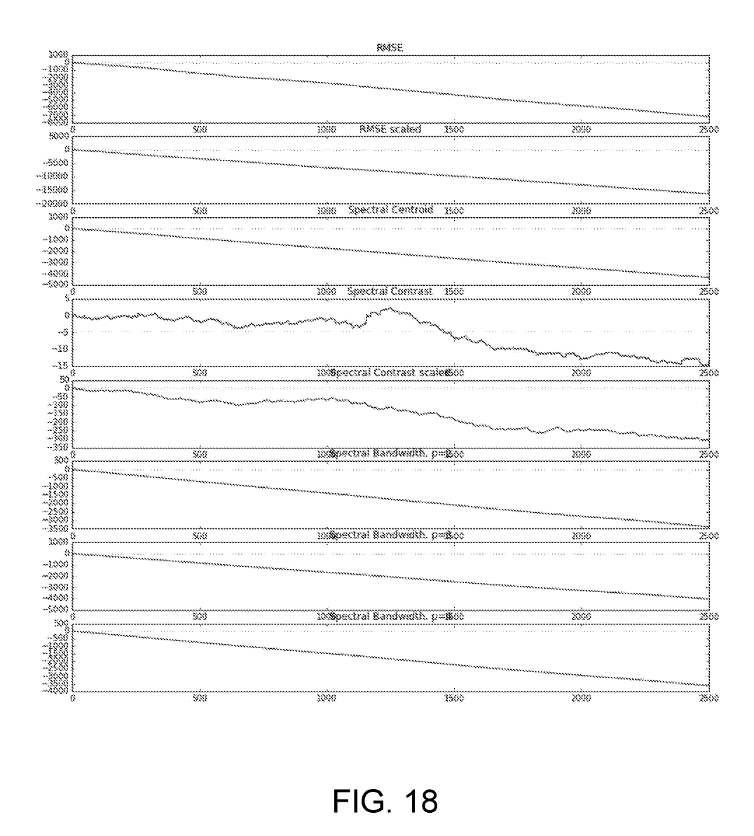

The code at In[20] computes and produces visualizations that distinguish the disgust emotional state from the having eaten gut state. The cumulative scores illustrated in FIG. 18 compare disgust to having eaten, for samples captured truly from having eaten. As shown, distinguishing disgust from having eaten is a significantly harder problem for two reasons. First, we have less information in each feature. Second, there are strong changes in the distributions over time (indicated by the slowly oscillating scores).

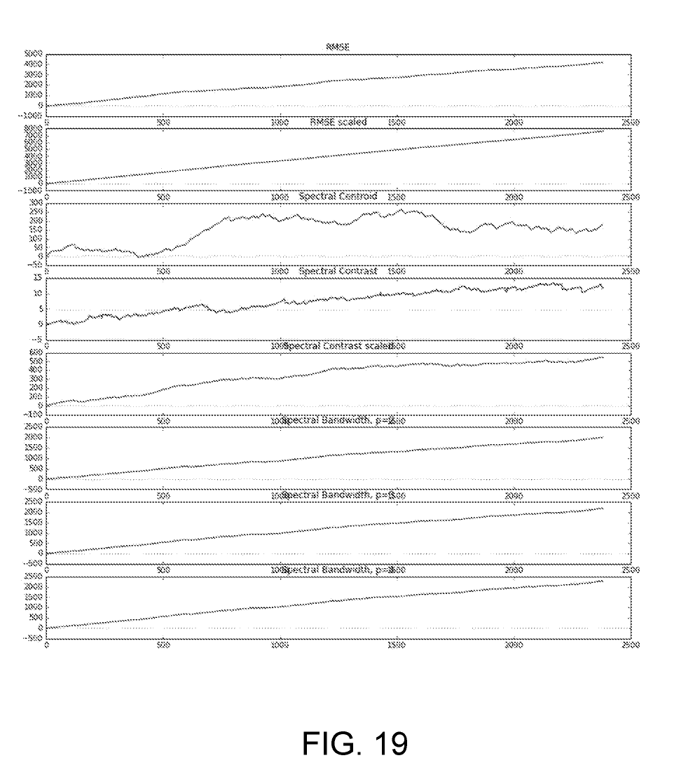

FIG. 19 shows visualizations for a comparison for samples that are actually from the disgust class (produced by the code at In[21]). The classification appears much easier here. This suggests that the disgust class could have a lot of overlap with the after eating class, but then include some additional behavior not observed in the after eating class.

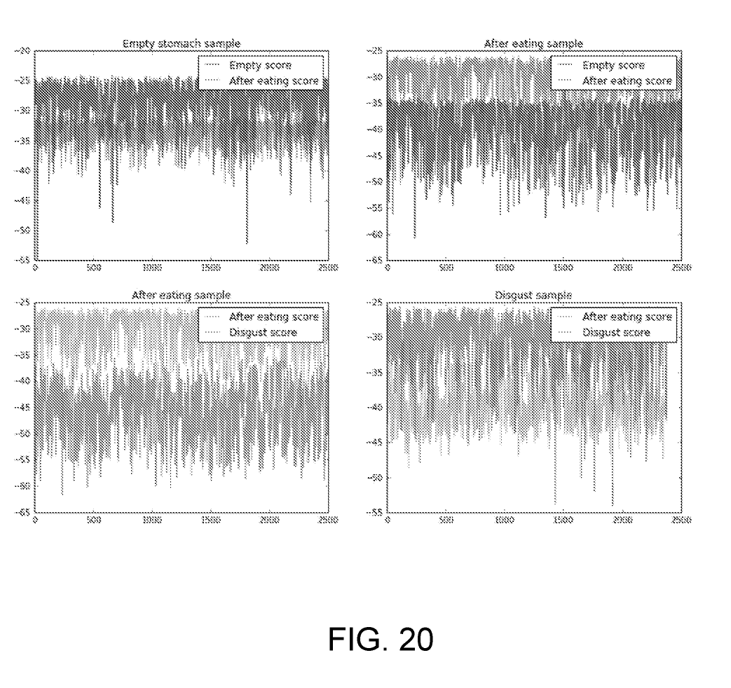

By combining the individual feature scores generated as described above into a single instantaneous score (In[22]) the visualizations of FIG. 20 are generated.

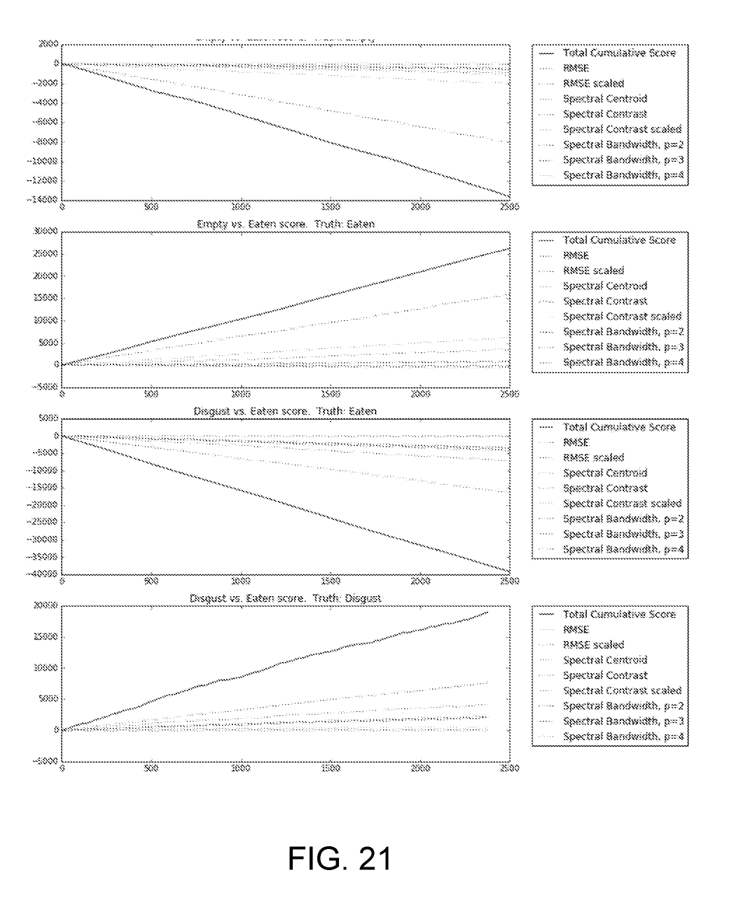

Then In[23] produces FIG. 21 which shows the cumulative differences broken down between classes and break outs of the total scores by the component feature scores to illustrate the driving factors.

The visualizations demonstrate that the features selected can provide lots of classification power. (Scores of around 20 are significant.) The slopes of the lines gives some indication how long the time periods need to be for effective classification.

At In[3] above reference is made to the following code (% run code/kde_models.py):

```
import soundfile as sf
import numpy as np
import librosa
import matplotlib
import matplotlib.pyplot as plt
from scipy import signal as sig
from scipy import stats
import copy
from cycler import cycler
def get_kernels(features):
    kernels = list( )
    for i, feat in enumerate(features[‘features’]):
        islog = features[‘feat_log’][i]
        if islog:
            kernels.append(stats.gaussian_kde(np.log(feat)))
        else:
            kernels.append(stats.gaussian_kde(feat))
    return(kernels)
# combine features across segments before doing kde
def get_kernels_multi(features_list):
    features = copy.deepcopy(features_list[0])
    for feat_set in features_list[1:]:
        for j, feat in enumerate(feat_set[‘features’]):
            features[‘features’][j] =
np.append(features[‘features’][j], feat)
    return(get_kernels(features))
def eval_kernel_logprobs(features, kernels):
    eps = 0.0001#Minimum probability, for dealing with zeros
    probs = list( )
    for i, feat in enumerate(features[‘features’]):
        islog = features[‘feat_log’][i]
        if islog:
            probs.append(np.log(eps+kernels[i].evaluate(np.log(feat))))
        else:
            probs.append(np.log(eps+kernels[i].evaluate(feat)))
    return(probs)
def plot_features_with_kernels(features, kernels):
    n_feat = len(features[‘features’])
    fig, axes = plt.subplots(nrows = n_feat, ncols=1, sharex=False,
sharey=False,figsize=(14, 14))
    for i, feat in enumerate(features[‘features’]):
        x = np.log(feat) if features[‘feat_log’][i] else feat
        plt.subplot(axes[i])
        x_grid = np.linspace(np.min(x), np.max(x), 1000)
        pdfs = kernels[i].evaluate(x_grid)
        #made Gray by Ali
        plt.hist(x,100, normed=True, color=(0.2,0.2,0.2))
        #changed to Grey by Ali
        plt.plot(x_grid, pdfs,color=‘gray’)
        title = features[‘feat_titles’][i]
        plt.title(title,color=‘gray’)
    plt.show( )
def compare_kernels(kernel_list, feature_list):
    plt.rcParams[‘axes.prop_cycle’] = plt.cycler(color=[(0.1, 0.1,
0.1),(0.5, 0.5, 0.5), (0.9, 0.9, 0.9)],linestyle=[‘:’, ‘--’, ‘-’])
    n_kern = len(kernel_list)
    n_feat = len(kernel_list[0])
    maxs = np.zeros(n_feat)
    mins = np.zeros(n_feat)
    for i, flist in enumerate(feature_list):
        for j, x in enumerate(flist[‘features’]):
            maxs[j] = np.max(x) if i==0 else max(np.max(x),maxs[j])
            mins[j] = np.min(x) if i==0 else min(np.min(x),mins[j])
    fig, axes = plt.subplots(nrows = n_feat, ncols=1, sharex=False,
sharey=False,figsize=(14, 14))
    #added by ali for gray
    #plt.rc(‘axes’, prop_cycle=(cycler(‘color’, [(0.2, 0.2, 0.2),(0.4,
0.4, 0.4), (0.6, 0.6, 0.6)]))
    #plt.axes.Axes.set_color_cycle([(0.2, 0.2, 0.2),(0.4, 0.4, 0.4),
(0.6, 0.6, 0.6)])
    for i in range(n_feat):
        if feature_list[0][‘feat_log’][i]:
            mins[i] = np.log(mins[i])
            maxs[i] = np.log(maxs[i])
        plt.subplot(axes[i])
        x_grid = np.linspace(mins[i], maxs[i],1000)
        for j in range(n_kern):
            pdfs = kernel_list[j][i].evaluate(x_grid)
            #color changed by ali
            plt.plot(x_grid, pdfs)
            title = feature_list[0][‘feat_titles’][i]
            plt.title(title)
    plt.show( )
# compare across people
def grand_compare(kernel_structure, feature_structure, title=“test”):
    a, *b = kernel_structure
    n_kern = len(a)
    n_feat = len(a[0])
    n_col = len(kernel_structure)
    maxs = np.zeros(n_feat)
    mins = np.zeros(n_feat)
    for k, feature_list in enumerate(feature_structure):
        for i, flist in enumerate(feature_list):
            for j, x in enumerate(flist[‘features’]):
                maxs[j] = np.max(x) if (i==0 and k==0) else
max(np.max(x),maxs[j])
                mins[j] = np.min(x) if (i==0 and k==0) else
min(np.min(x),mins[j])
    fig, axes = plt.subplots(nrows = n_feat, ncols=n_col, sharex=False,
sharey=False,figsize=(24, 14))
    fig.suptitle(title, size=20)
    for k in range(n_col):
        for i in range(n_feat):
            if (feature_structure[k][0][‘feat_log’][i] and k==0):
                mins[i] = np.log(mins[i])
                maxs[i] = np.log(maxs[i])
            plt.subplot(axes[i,k])
            x_grid = np.linspace(mins[i], maxs[i],1000)
            for j in range (n_kern):
                pdfs = kernel_structure[k][j][i].evaluate(x_grid)
                #color changed to grey by ali
                plt.plot(x_grid, pdfs,color=‘gray’)
                title = feature_list[0][‘feat_titles’][i]
                plt.title(title)
    plt.show( )
```

Although the code, text, and visualizations we have discussed are directed to understanding the effectiveness of the classification of features of gut sounds among gut states and emotional states, similar code and sample processing and the results of it can be used in many other applications.

Although the examples refer to certain features of the acoustic samples acquired during states related to eating and disgust, samples of gut sounds can be acquired in connection with a wide variety of other gut states and emotional states and then be classified into gut states and emotional states.

Also, although we have described certain features of the acoustic samples as useful for classification, a wide variety of other features may also be useful, for example, features that reflect sonic structure at higher temporal levels like similarity matrices, rhythmic analysis, meter detection, and genre classification. Highly informative features can also be adaptively estimated directly from the training data, as an alternative to standard features from audio analysis.

Also, although we have described examples in which a non-parametric naïve Bayes model is used to capture feature distribution, other models can be used including more complicated and perhaps more effective models including other distribution models and other classifiers. The technology that we have described is not restricted to using a naïve (independence) model for the features or over time points.

Therefore, we have described a device that measures acoustic gut activity and methods, which use machine learning, to process and interpret the acoustic signals produced by the device. The device and methods can be used to examine the determinants of gut activity and activity-suppression, identify gastric disorders on the basis of sounds, as a therapeutic tool for enabling gut regulation via biofeedback.

In applying the technology that we describe here, input from a first study was used to determine the positioning of the microphones. Then we induced states such as stress and disgust in subjects to see whether we could determine the timing and nature of these inductions on the basis of gastric sounds. This is a key study for determining the potential usefulness of the apparatus in future studies examining the environmental and psychological determinants of gastric activity.

In another use of the technology that we describe here, we recruited gut-healthy subjects and subjects suffering from inflammatory bowel disease (IBD) to determine the extent to which we can discriminate between the two groups on the basis of sound analysis.

An embodiment of the technology includes a robust and mass-producible device suited for psychogastroenterology data collection and analysis.

Studies using the devices could be case-control studies that gather data from some people who are controls (e.g., not sick) and some who are cases (e.g., sick), usually out of the proportion in the base population. Non-case-control data settings have yielded complex data, consisting of multivariate time series and spectral data from source devices with uncertain placement. Initial analysis of the data suggested that the signal was present at a variety of time scales and frequencies. The novelty of the recording approach and the complexity of the data lend themselves to flexible machine learning approaches that detect structure and automate classification.

We use statistical machine learning approaches for feature extraction and selection, driven by differences in the case-control populations, to construct specialized features adapted to this data. This allows identification of particular features of the signal that are strongly associated with eating, stress, and IBD. We also develop classification procedures that use these features to distinguish between the various types of gastrointestinal activity we have measured.

With an eye to clinical application, we combine the rough estimates available from these small pilot studies with base rate information from the literature, to obtain estimates of the potential for these features and classifiers to be informative as clinical measures.

In addition to the classification procedures, the discovered features are informative in their own rights. We examined the consistency of meaningful features across experimental settings, investigating whether the acoustic features that are effective for detecting IBD are also effective for recognizing stress or eating states. This gives insight into the structure of the underlying problem, and gives some indication of the extensibility of our features and methods to other gastrointestinal settings. The features may also reveal details about the underlying science; if durations of certain events are found to be informative in IBD, this may be revealing about the underlying digestive process.

The device and its use can be taken in multiple directions. At both a research and commercial level, the device can be used as a tool for providing biofeedback to IBD patients to help them to regulate their conditions. At a more academic level, the existence of a non-intrusive tool for measuring gastric activity, including in daily life, can kick-start the new field of psychogastroenterology.

Among other uses, the acoustic signals and features captured and classified by the system may be used (as mentioned earlier) as bio-feedback to the user to capture, for example, episodic gastrointestinal events and provide indications of triggering physical or emotional states. The system can capture continuous signals over long periods of time that may suggest dietary or behavioral modifications effective to regulate gut function (gut state) and reduce episodic events.

In clinical settings, the capture of gut sound acoustic samples, by either or both episodic and continuous monitoring, may be integrated into systems for clinical management of patients having a broad variety of gastrointestinal maladies. Patient data (including and in addition to the acoustic samples and results of analyzing them) may inform the therapy approaches for physiological management of the digestive tract and behavioral therapies to manage emotional states that trigger inflammation and gastrointestinal events.

The system may also present information that will be useful in the management, discharge, and monitoring of post-surgical patients experiencing (common) gut paralysis, for which motility, measured acoustically, is a desired milestone in the recovery as well as the avoidance of readmission.

Gut sound acoustic samples and related data can be aggregated across patients and other users and can be applied in research in the fields of gastroenterology and the emergent space of pyscho-gastroenterology. The uses can involve in fundamental discovery and pre-clinical trials for patient segmentation for use in clinical trials of pharmaceuticals for gastric disease, neurological disorders, and cancer therapies.

The sensor device 102 (FIG. 1) can be implemented in a wide variety of ways. In some implementations (FIG. 22), the sensor device includes a belt 200 to be worn around the waist of the subject and two high quality stethoscope microphones 204 configured so that when the belt is worn the microphones are inwardly facing toward the skin of the abdominal skin of the subject. Cables 206 provide external connections direction to the microphones for coupling to a recorder (not shown).

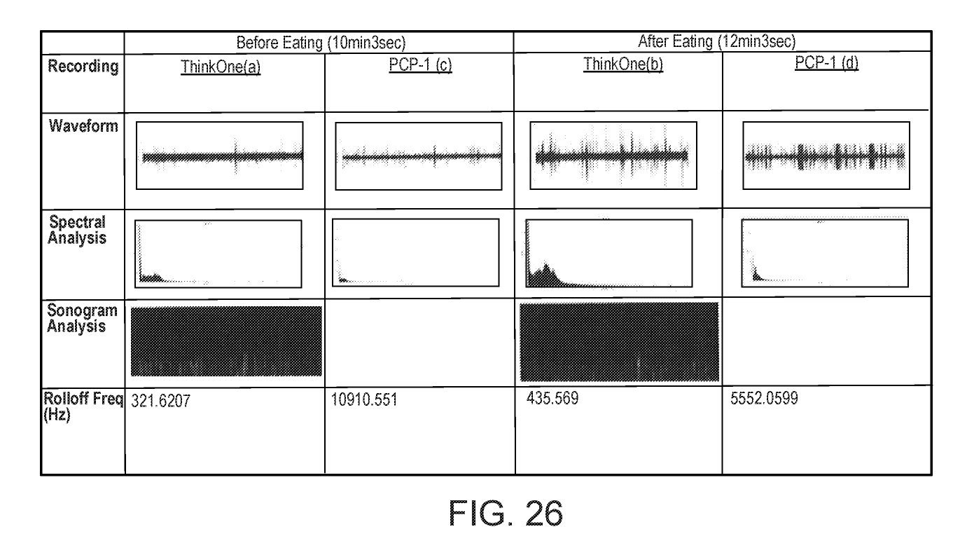

We constructed five of the belt shown in FIG. 1 and then made ten 2.5-hour recordings of different people's gut activity (gut state), beginning after an extended period of fasting, and with an ingestion of food beginning exactly one hour into the recording. The data from these recordings was analyzed using spectral analysis. Initial analyses are encouraging; for example, as shown in FIG. 26, which displays spectral analyses of one subject's data, showing 30 minutes (1800 samples) per panel from bottom to top in FIG. 26. The data clearly show an increase in activity beginning early in the second hour, the time at which the subject began ingesting food.

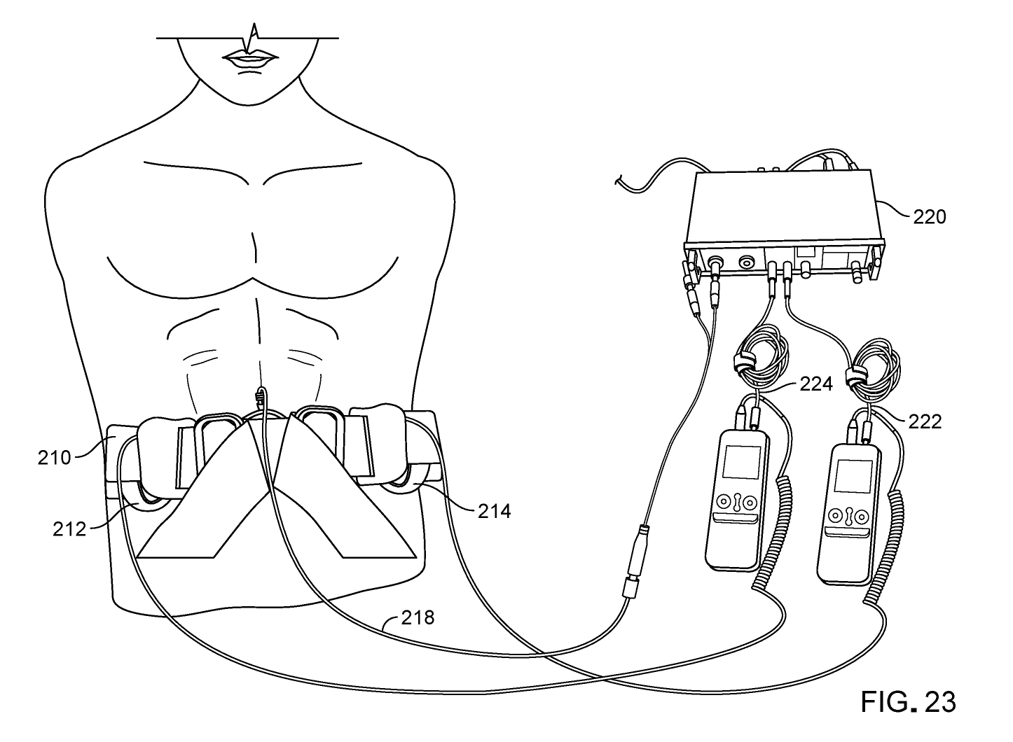

As shown in FIG. 23, another prototype that uses an innovative electronic stethoscope (ThinkOne) was developed that captures audio with much greater fidelity. Using this prototype, we performed a simple experiment to compare audio recordings of the gut before and after a meal; the experiment was successful. Before-and-after-eating audio recordings were substantially different in ways that could be readily detected with spectral analysis. In FIG. 23, a belt 210 bears inwardly facing microphones 212, 214. A cable 218 carries the output signals to an audio interface 220. Also coupled to the audio interface are the outputs 222 and 224 from two preamps that receive input signals from the two microphones 212, 214.

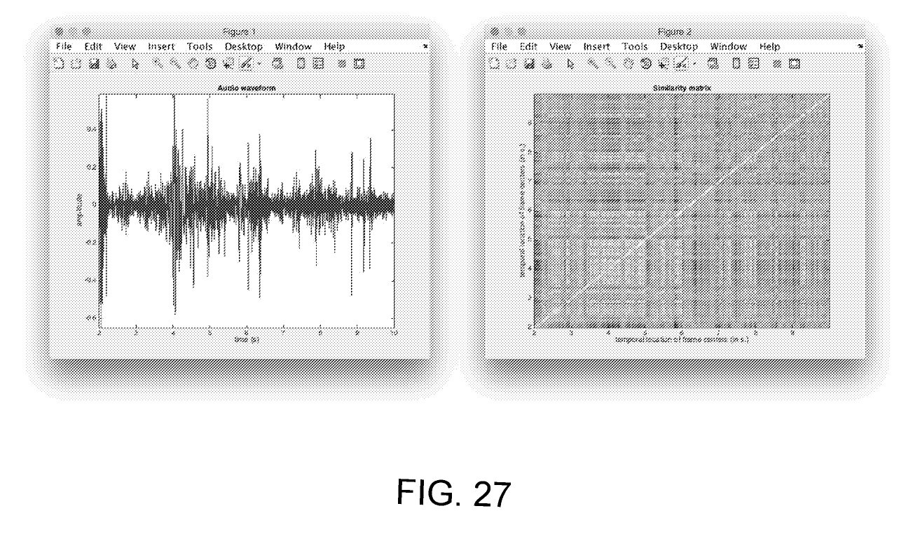

FIG. 27 illustrates differences in quality of outputs using the microphones of the first prototype and the ThinkOne microphone.

FIG. 28 illustrates a raw audio waveform from the ThinkOne microphone and a similarity matrix that portrays a comparison between each moment of sound in a recording sample, with every other moment of sound in the same recording to arrive at a measure of how the audio sample changes over time and how similar or dissimilar different moments of an audio sample are from the others; this analysis can reveal temporal structure in an audio sample beyond what is possible to calculate through extracted spectral features

There can be numerous sources of unwanted additive noise in the recordings including heartbeats, ambient noise, and vocalizations by the wearer of the belt. The intrinsic signal quality of the signals from the microphones can be improved substantially by a judicious combination of state-of-the-art fixed and adaptive de-noising and noise-canceling algorithms, the latter taking advantage of additional microphones far from the gut to provide signal-free reference channels.

In another prototype approach for a wearable gastro-listening instrument, we use three of the microphone belts simultaneously (six microphones), as well as one outward facing microphone (employed in the analysis to cancel out ambient noise), and use a high quality multi-channel recorder. We recorded another 5 subjects, again for 2.5 hours each with a meal consumed one hour into the session. Six microphones permit a much more comprehensive, higher quality, input into new, more sophisticated, statistical analyses.

The number of microphones and the pattern in which they are placed can be determined empirically based on the quality of results produced by the corresponding acoustic signals and the features that can be derived from them and classified effectively. Using larger numbers of microphones may yield more or better information about the spatial characteristics of the gut sounds.

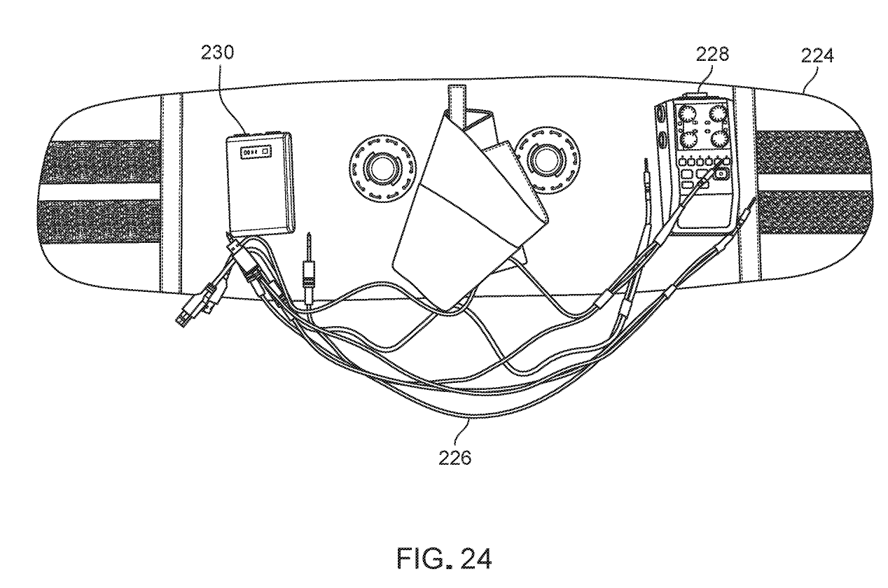

In another prototype, shown in FIG. 24, a neoprene belt 224 carries two ThinkLabs microphones facing inwardly (into the page) coupled by cables 226 to a four-channel recorder 228, mounted on the outwardly facing side of the belt. A battery pack 230 powers the recorder. Because the belt is “self-powered” it can be worn for longer periods and while the subject is doing other things.

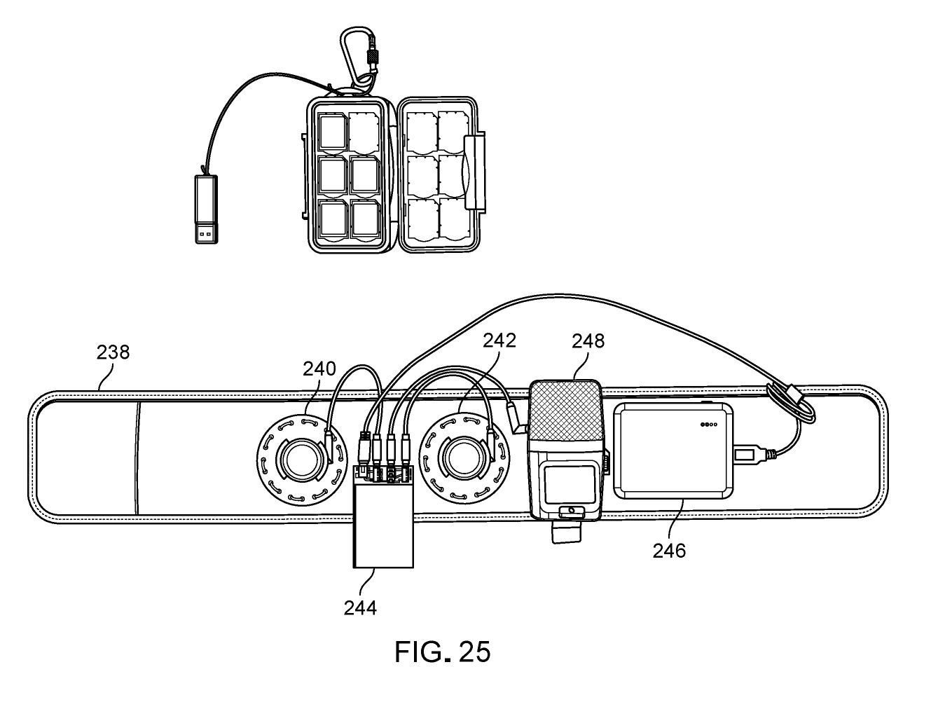

In another prototype, shown in FIG. 25, the two ThinkOne stethoscope microphones 240, 242 are mounted in 3D printed ABS plastic holders hand sewn to a neoprene belt 238. The microphones are coupled by TRS cables to a custom circuit board 244 mounted on the outwardly facing side of the belt. The custom circuit board sends power from a battery 246, also mounted on the outwardly facing side, to the stethoscopes and combines audio from the two stethoscopes into a stereo output that can be delivered through a TRS coupler to a four channel audio recorder 248 that records the signals from the two stethoscopes as well two directional microphones that capture ambient sounds in the room. The audio recorder records onto SD cards 248 which can then be read by an SD card reader through a USB port.

Whatever recording device is used, the gut sounds are captured as time series of digitized acoustic samples for later processing and analysis as discussed earlier.

In some instances it would not be necessary to record the acoustic samples on the belt or through a cable to a nearby recorder. In some cases, the belt could carry only the microphones and a transmitter to send the signals wirelessly to a local device such as a mobile phone where the acoustic samples could be stored or (without storing them) forwarded to cloud-based storage.

Recording Protocol

For purposes of developing sets of training data (or real data to be analyzed to identify gut states or emotional states) using carefully controlled conditions, a protocol such as the following can be applied.

1. A survey questionnaire including questions about digestive conditions, bowel movements, and menstrual cycle.

2. A baseline recording of ten minutes of, e.g., reading a book.

3. A recording of twenty minutes associated with disgust-eliciting film/audio clips.

4. Five minutes in a resting state.

5. A ten-minute preparation period in which the subject hears amplified ambient noise made by the subject.

6. A period of five minutes of speech followed by five minutes of resting with the speech period not being recorded.

7. Ten minutes recording a concentration task such as playing Tetris.

8. Ten minutes recording a frustration task such as playing a modified Tetris game.

9. Ten minutes recording eating food (e.g., a sandwich) followed by 35 minutes of digestion.

Biofeedback

In some applications, the system can be applied to enable users to develop an implicit or explicit understanding (and control) of their own gut functioning (gut state) and of the factors that moderate the gut state.

In some implementations, a mobile-phone-based system could present a user with real-time data visualizations (graphics) and sonifications (sounds) based on and representative of acoustic samples of gut activity (gut state). To create such visualizations and sonifications of gut activity (gut state), the system can continuously capture acoustic samples of gut sounds. The system could also continuously capture motion sensor data that reflects the user's physical state (posture, movements) and environment (temperature, humidity, pressure). The system can extract features from the acoustic samples and classify features and sets of features as markers for specific gut states. The system would employ this analysis to create didactic (charts, plots, graphs, sine tones, and noise, for example) and aestheticized (geometric patterns, abstract blobs, color fields, musical melodies and harmonic textures, among others) visuals and sounds to convey gut activities (gut states) to users in a different, instantly comprehensible, format. The feedback system would be complemented by guidance, presented either by a coach or online interface, about different strategies that can be employed to regulate gut function (gut state), including physical or mental activities such as relaxation, meditation, mindfulness or mental exercises.

In such a system, a mobile phone could be provided with an interactive user interface for controlling the capturing of acoustic samples of gut sounds. The continuous monitoring of the user's physical state could include monitoring when, for example, the user is standing or reclining, moving, or has been standing for some long time period, such as four hours. Monitoring of environmental conditions can include for example whether it's cold or raining).

The user interface could present real-time data visualization of gut activities (gut state) based on analyzed features and gut state classifications. Visualization in the user interface could also include a representation of the user's gut health in the form of a continuously evolving avatar with which users develop an ongoing relationship.

Experience Sampling

As mentioned earlier, experience sampling can be combined with the capturing and use of acoustic samples representing gut sounds. Such a system could include a mobile phone, a wearable device to sense the gut sounds, and a mobile app that reminds users at certain times to record their thoughts, activities (including, potentially, whom they are with), and feelings at the moment in time. The system can integrate this data with the information provided by the continuous audio recordings of gut function (gut state). In some implementations, the wearable device would be designed for long term use. The system could conduct time-based statistical analysis to estimate causal and correlational relationships between gut function (gut state) and emotional states (such as thoughts or feelings) and between gut states and activities engaged in by the user. Such a system can be used for a wide variety of applications and purposes, including research. In some applications, the system would provide feedback to consumer users about their personalized idiosyncratic connections between their thoughts, feelings, activities and gut activity (gut state).

Crowdsourcing Gut Wisdom

In some applications, a system could include a mobile phone, a wearable device, and a mobile app configured to crowdsource gut wisdom. The system would accumulate and maintain a large (e.g., cloud-based) database of acoustic samples of gut sounds of a large population of users, and a corresponding database of the users' self-reported intestinal disorders, if any, and, in data collected over time and merged with gut sound data, self-reported thoughts, feelings (e.g., emotional states), and activities, using experience sampling techniques and captured through the mobile phone app. The system could apply classification and regression algorithms to identify relationships between thoughts/feelings/physical/emotional states and gut function (gut states) across large populations. Using these algorithms, the system would (1) make suggestions to users about how they may be able to better understand or control their gut functions (gut states), (2) give users clues about future psychosomatic events (e.g., migraines), based on their current gut state, (3) give users clues about their future gut state based on information about their activities, thoughts, and feelings (e.g. emotional state), and (4) give users advice about how to change their lifestyle, including diet and activities, to improve gut function (gut state).

In some implementations, users who buy the wearable device would receive the app free, and, in exchange for providing demographic and health-related information including intestinal disorders as well as ongoing experience sampling and gut sound data, would receive the personalized information mentioned above.

Other implementations are also within the scope of the following claims.

For example, in some cases, any one or more of the following activities can be performed at a cloud based server system: analysis, comparison, classification, recognition, storage, web serving, account maintenance, and others.

The sensing device and other elements of the system can be designed to be worn, used, or operated continuously for long periods of time, for example, many days, without interruption, enabling more complex and productive analysis and results to be developed and provided to users.

Regardless of the length of time of continuous use, the sensing device can be configured to be worn comfortably during normal activities such as walking, working, or eating.

For purposes of inducing particular emotional states, multimedia stimulus elements and content can be developed, stored, and applied to users.

Among the diagnostic and therapeutic applications are recognizing and treating post-operative ileus and recognizing and treating gastro-intestinal blockages.

The machine learning techniques used by the system need not be limited to Bayesian classifiers, and could include other approaches such as deep neural networks.

1. An apparatus comprising
a sensor configured to sense sound from a gut of a person and to output a corresponding acoustic signal,
a signal processor having the acoustic signal as an input and configured to output corresponding digital acoustic samples,
a storage device for the digital acoustic samples,
a processor configured to apply the digital acoustic samples to a model to determine an aspect of a state of the person, and
a device configured to present information about the determined aspect of the person's state to a user.

2. The apparatus of claim 1 in which the sensor comprises one or more microphones or stethoscopes.

3. The apparatus of claim 1 comprising a structure to be worn by the person and to support the sensor in an orientation to receive the sound from the gut.

4. The apparatus of claim 3 in which the structure to be worn by the person also supports a wireless transmitter.

5. The apparatus of claim 1 in which the model comprises a predictive model.

6. The apparatus of claim 5 in which the model comprises a Bayesian model.

7. The apparatus of claim 1 in which the aspect of the state of the person comprises a gut state or an emotional state.

8. The apparatus of claim 1 in which the information presented to the user comprises a visualization associated with features of the acoustic samples.

9. The apparatus of claim 1 in which the information presented to the user comprises audiovisual representations of the determined aspect of the person state.

10. The apparatus of claim 9 in which the audiovisual representations comprise biofeedback for the user.

11. The apparatus of claim 1 in which the information presented to the user is representative of a diagnosis of the aspect of the state of the person.

12. The apparatus of claim 1 in which the information presented to the user is representative of a therapy for the aspect of the state of the person.

13. The apparatus of claim 1 in which the information presented to the user is representative of a prognosis for the aspect of the state of the person.

14. A method comprising
sensing sound from a gut of a person,
storing digital acoustic samples representative of the sound from the gut of the person,
predicting an aspect of a state of the person based on the stored digital acoustic samples, and
presenting information about the determined aspect of the person's state to a user.

15. The method of claim 14 comprising sensing the sound from the gut of the person through the skin of the abdomen of the person.

16. The method of claim 14 in which the predicting comprises applying information about the digital acoustic samples to a predictive Bayesian model.

17. The method of claim 14 in which the aspect of the state of the person comprises a gut state or an emotional state.

18. The method of claim 14 in which the presenting of the information to the user comprises presenting a visualization associated with features of the acoustic samples.

19. The method of claim 14 in which the presenting of the information to the user comprises presenting audiovisual representations of the determined aspect of the person state.

20. The method of claim 14 comprising diagnosing the aspect of the state of the person.

21. The method of claim 14 comprising identifying a therapy for the aspect of the state of the person.

22. The method of claim 14 comprising providing a prognosis for the aspect of the state of the person.

23. The method of claim 14 comprising sampling an experience of the person associated with the digital acoustic samples.

24. An apparatus comprising
a device configured to sense sound from a gut of a person and to store digital acoustic samples corresponding to the gut sound,
a processor configured to determine an aspect of a state of the person based on the stored digital acoustic samples, and
a device configured to feed back representations of the determined aspect of the person's state to the person.

25. The apparatus of claim 24 in which the representations are configured to enable the person to understand or control the person's gut state or contributors to the gut state.

26. The apparatus of claim 25 in which the representations comprise audiovisual abstractions.

27. The apparatus of claim 26 in which the audiovisual abstractions comprise sonifications or visualizations corresponding to the state of the person.

28. The apparatus of claim 24 in which the device is configured to be worn by the person continuously and the digital acoustic samples are stored continuously.

29. The apparatus of claim 24 comprising
a device configured to sense a physical state of the person or of the person's environment.

30. The apparatus of claim 29 in which the aspect of the state of the person is based also on the physical state of the person or of the person's environment.

31. The apparatus of claim 24 in which the fed back representations comprise didactic or aestheticized audiovisual elements.

32. The apparatus of claim 24 in which the representations fed back to the person comprise diagnoses, prognoses, or therapies.

33. A method comprising
sensing sound from a gut of a person and storing digital acoustic samples corresponding to the gut sound,
determining an aspect of a state of the person based on the stored digital acoustic samples, and
feeding back representations of the determined aspect of the person's state to the person.

34. The method of claim 33 in which the representations are configured to enable the person to understand or control the person's gut state or contributors to the gut state.

35. The method of claim 34 in which the representations comprise audiovisual abstractions.

36. The method of claim 35 in which the audiovisual abstractions comprise sonifications or visualizations corresponding to the state of the person.

37. The method of claim 35 in which the device is worn by the person continuously and the digital acoustic samples are stored continuously.

38. The method of claim 35 comprising
sensing a physical state of the person or of the person's environment.

39. The method of claim 38 in which the aspect of the state of the person is based also on the physical state of the person or of the person's environment.

40. The method of claim 33 in which the fed back representations comprise didactic or aestheticized audiovisual elements.

41. The method of claim 33 in which the representations fed back to the person comprise diagnoses, prognoses, or therapies.

42. An apparatus comprising
a device configured to sense sound from a gut of a person and to store digital acoustic samples corresponding to the gut sound,
a device configured to receive information representing an experience of the person corresponding to the gut sound,
a processor configured to determine an aspect of a state of the person based on the stored digital acoustic samples and to determine a correlation between the aspect of the state of the person and the experience of the person, and
a device configured to present information about the determined aspect of the person's state and the determined correlation to a user.

43. A method comprising
sensing sound from a gut of a person and storing digital acoustic samples corresponding to the gut sound,
receiving information representing an experience of the person corresponding to the gut sound,
determining an aspect of a state of the person based on the stored digital acoustic samples and determining a correlation between the aspect of the state of the person and the experience of the person, and
presenting information about the determined aspect of the person's state and the determined correlation to a user.

44. An apparatus comprising
a central server configured to receive, from devices of a population of users, data representing gut sounds of each of the users and experiences of each of the users associated with the user's gut sounds, and
a processor configured to analyze the gut sounds and experiences of the population and to output inferences about correlations between the gut sounds in the experiences.

45. The apparatus of claim 44 in which the experiences comprise self-assessed experiences.

46. The apparatus of claim 44 in which the experiences comprise one or more of thoughts, feelings, emotional states, or activities.

47. The apparatus of claim 44 in which the processor is configured to apply classification or regression to identify relationships between the experiences and gut states represented by the gut sounds.

48. The apparatus of claim 44 in which the processor is configured to output suggestions about how to understand or control gut state.

48. The apparatus of claim 44 in which the processor is configured to output predictions about future states of a person.

49. A method comprising
at a central server, receiving, from devices of a population of users, data representing gut sounds of each of the users and experiences of each of the users associated with the user's gut sounds, and
analyzing the gut sounds and experiences of the population and outputting inferences about correlations between the gut sounds and the experiences.

50. The method of claim 49 in which the experiences comprise self-assessed experiences.

51. The method of claim 49 in which the experiences comprise one or more of thoughts, feelings, emotional states, or activities.

52. The method of claim 49 comprising applying classification or regression to identify relationships between the experiences and gut states represented by the gut sounds.

53. The method of claim 49 comprising outputting suggestions about how to understand or control gut state.

54. The method of claim 49 comprising outputting predictions about future states of a person.
